(19) United States
(12) Patent Application Publication
(10) Pub. No.: US 2026/0337663 A1
(43) Pub. Date: Oct. 29, 2026

(54) DETECTION OF TARGET ANALYTES USING MULTIMODAL SIGNAL PROBES

(71) Applicant: Roche Molecular Systems, Inc., Pleasanton, CA (US)

(72) Inventors: Jenise Boey Chen, Vista, CA (US); Edward H. Cho, San Diego, CA (US); Alden Christian Moss, Del Mar, CA (US); Ali Sepehri Fard, Carlsbad, CA (US); Ankur H. Shah, San Diego, CA (US); Kunal Sur, San Diego, CA (US); Uichong Brandon Yi, San Diego, CA (US)

(21) Appl. No.: 19/484,121

(22) PCT Filed: May 13, 2024
(86) PCT No.: PCT/EP2024/063075

§ 371 Date: Nov. 13, 2025

Related U.S. Application Data
(60) Provisional application No. 63/466,866, filed on May 16, 2023.

(51) Int. Cl. C12Q 1/6816

(52) U.S. Cl. CPC C12Q 1/6816

(57) ABSTRACT

Disclosed is a dual-labeled signal probe comprising an electron transfer moiety (ETM) and an optical signaling moiety (OSM). Upon binding of a target analyte to the signal probe, the composition is transported to an electrode surface. In some embodiments, the ETM and OSM are then each excited by applying an electrical voltage to the electrode. In some embodiments, the ETM signal and OSM signal are then detected using a single detector (or more than one detector), allowing the presence or absence of the target analyte to be determined. In some embodiments, the ETM signal and OSM signal are correlated before determining the presence or absence of the target analyte. In some embodiments, the dual-labeled signal probe permits real-time detection using an end-point electrochemical detection system. In some embodiments, the dual-labeled signal probe may be used to assess the location and density of capture probes on an electrode.

### FIELD OF THE INVENTION

The invention relates to the field of molecular diagnostic methods and particularly to the detection of one or more analytes, including biomolecules in an obtained sample.

### BACKGROUND OF THE INVENTION

Bloodstream infections (BSIs) are associated with significant morbidity, mortality, and increased length of stay (LOS). Delayed administration of effective antibiotics increases the risk of mortality, and therefore correct selection of an antibiotic regimen early in the treatment process is paramount. Delayed identification of the causative organism and antimicrobial resistance or susceptibility may often be responsible for delays in optimal antimicrobial therapy. Rapid diagnostic testing (RDT), which includes tests, such as polymerase chain reaction (PCR), matrix-assisted laser desorption/ionization-time of flight mass spectrometry (MALDI-TOF MS), and peptide nucleic acid fluorescent in situ hybridization (PNA-FISH), has improved upon conventional microbiological methods, reducing time to organism identification, optimizing antimicrobial therapy, and subsequently improving clinical outcomes, including mortality. Commercial molecular RDTs (mRDT) are available for direct testing of positive blood culture bottles (BCBs), providing timelier results than conventional subculture and phenotypic susceptibility testing. Examples of commercially available mRDTs devices include Applicant's ePLEX® diagnostic system.

Electrochemical detection systems are valuable tools, which are highly sensitive and can detect small amounts of target. Electrical and electrochemical monitoring of nucleic acid amplification requires no optical assistance so that the system can be simplified, downsized, and integrated into a small chip with the aid of complementary metal oxide semiconductor (CMOS)-compatible fabrication process, leading to the production of a scalable high-throughput analysis system in point-of-care applications.

In these electrochemical systems, the target amplicons are typically mixed with ferrocene-labeled signal probes (or osmium-labeled signal probes) that are complementary to the specific targets on the panel. Target sequences hybridize to the complementary signal probe and capture probes, which are bound to gold-plated electrodes, as shown in FIG. 1a. The presence of each target is determined by voltammetry, which generates specific electrical signals from the ferrocene-labeled signal probe (or the osmium-labeled signal probe). The use of microfluidic systems in the electrochemical detection of target analytes is described in more detail in U.S. Pat. Nos. 10,005,080, 9,557,295, 8,501,921, 6,600,026, and 6,740,518, the disclosures of which are herein incorporated by reference in their entireties.

A potential drawback to electrochemical detection technology is that it relies on end-point analysis, which is disadvantageous because (1) the system requires post-amplification processing adding time before a detection result is achieved; (2) quantification is difficult because of the narrow dynamic range compared to fluorescence-based real-time PCR methods. Thus, there remains a need to further improve electrochemical detection systems.

Fluorescence-based bioassays are believed to lack sensitivity and to require expensive optical instruments. In addition, the biorecognition events in these assays are inherently slow (several minutes to hours). The sensitivity of the fluorescence-based assays can be improved, without the use of high-end optical instruments, by incorporating plasmon resonant particles (PSPs). It is believed that the improved sensitivity is made possible by the increase in fluorescence signatures and decreased lifetimes of fluorophores placed in close proximity to PSPs, described by a phenomenon called Metal-Enhanced Fluorescence (MEF). In MEF-based bioassays, PSPs (generally silver nanoparticles) are deposited onto the planar surface and the bioassay is constructed on the PSPs. Since the size of most biomolecules are smaller than PSPs (20-100 nm), fluorophores are positioned within a distance where their emission is increased due to their interactions with the surface plasmons of PSPs. Digital fluorescence detection is disclosed in U.S. Pat. No. 9,810,637, which is herein incorporated by reference in its entirety. In these MEF bioassays, the fluorophore is enhanced by a wavelength of light, not by electricity. Further, these MEF bioassays do not use a signal probe with both an electron transfer moiety (ETM) and an optical signaling moiety (OSM). Further, these MEF bioassays do not utilize a capture probe to hold the OSM in close proximity to the surface. Further, the goal in MEF bioassays is to eliminate the need for photo detectors to convert photon fluxes into digital signatures such as photomultiplier tube or charge coupled device (CCD) camera, which are still required under the disclosed methodologies.

### SUMMARY OF THE INVENTION

A first aspect of the present disclosure are signal probes including a first signaling moiety and a second signaling moiety, wherein the first signaling moiety and second signaling moiety have different detection modalities (referred to herein as a “dual-labeled signal probe”). In some embodiments, the first signaling moiety is an electron transfer moiety (ETM); and the second signaling moiety is an optical signaling moiety (OSM). In some embodiments, the dual-labeled signal probe is bound to a target analyte (e.g., a nucleic acid) to form a target/dual-labeled signal probe complex. In some embodiments, the target/dual-labeled signal probe complex is bound to a capture probe to form a capture probe-dual labeled signal probe-nucleic acid system complex. In some embodiments, the capture probe-dual labeled signal probe-nucleic acid system complex is bound to an electrode (“capture probe-signal probe-nucleic acid system complex”).

A second aspect of the present disclosure are compositions comprising (i) a first dual-labeled signal probe including a first signaling moiety having a first detection modality and a second signaling moiety having a second detection modality; and (ii) a second dual-labeled signal probe including a third signaling moiety having the first detection modality and a fourth signaling moiety having the second detection modality; wherein at least the signals produced by the second signaling moiety and fourth signaling moiety are distinguishable.

A third aspect of the present disclosure are one or more capture probe/stained DNA hybridization complexes, where each the one or more capture probe/stained DNA hybridization complexes comprise fluorescently stained nucleic acids on an electrode bound to a capture probe. In some embodiments, a voltage can be applied to the one or more capture probe/stained DNA hybridization complexes. In some embodiments, the fluorescently stained nucleic acid produces a detectable signal in response to the applied voltage.

In each of the compositions described above, a detectable signal may be detected by an optical detector, an electrochemical detector, or both an optical detector and an electrochemical detector. In this manner, a fluorescently stained nucleic acid (or an OSM on a signal probe) may be measured by application of a voltage. In some embodiments, the detectable signal is a dipole moment, plasmonic current flow, and/or fluorescing.

A fourth aspect of the present disclosure is a method of detecting a target analyte (e.g., a nucleic acid) in a sample. In some embodiments, the method comprises introducing a signal probe to an obtained sample including a target analyte, such that the target analyte binds to the signal probe (e.g., a dual-labeled signal probe) and a capture probe to form a capture probe-signal probe-target analyte system complex (e.g., a capture probe-dual labeled signal probe-target analyte system complex). In some embodiments, the signal probe is a dual-labeled signal probe comprising a first signaling moiety and a second signaling moiety, where the first and second signaling moieties have different detection modalities. In some embodiments, the first signaling moiety is an ETM; and the second signaling moiety is an OSM. In some embodiments, and following excitation, a signal from the first signaling moiety and a signal from the second signaling moiety are detected (either with a single detector or multiple detectors of different modalities). After detection, the signal from the first signaling moiety and the signal from the second signaling moiety are correlated as disclosed herein.

In some embodiments, an optical signal is detected by an optical detector following application of an applied voltage to the dual-labeled signal probe. In some embodiments, an optical signal is detected by an electrochemical detector following application of an applied voltage to the dual-labeled signal probe. In some embodiments, an optical signal is detected by an electrochemical detector and an optical detector following application of an applied voltage to the dual-labeled signal probe. Thus, the present disclosure provides for detecting optical signals such as colorimetric signals, fluorescence signals, luminescence signals, chemiluminescence signals and/or phosphorescence signals without the need for additional activators (such as light sources) to excite the label (and in some embodiments, without the addition of an optical detector).

A fifth aspect of the present disclosure is a method of detecting one of a redox reaction or an oxidation reaction, comprising: (a) introducing a signal probe to an obtained sample; (b) irradiating the obtained sample with light or a laser beam; and (c) detecting the one of the redox reaction or the oxidation reaction from one or more signaling moieties (e.g., an ETM or an OSM) coupled to the signal probe. For example, in some embodiments, a dual-labeled signal probe comprising first and second signaling moieties is introduced to an obtained sample, wherein the first signaling moiety is an ETM and wherein the second signaling moiety is an OSM. Subsequently, the obtained sample (including the introduced signal probe) is irradiated with electromagnetic radiation source (e.g., light or a laser beam); and a redox reaction or an oxidation reaction is detected. In some embodiments, the redox reaction or oxidation reaction is detected by an optical detector following the irradiation of the obtained sample with the light or the laser beam. In some embodiments, the redox or oxidation reaction is detected by an electrochemical detector following the irradiation of the obtained sample with the light or the laser beam. In some embodiments, a redox or oxidation reaction is detected by both an electrochemical detector and an optical detector following the irradiation of the obtained sample with the light or laser beam. Thus, the present disclosure provides for detecting ETM signals without the need for ETM activators (such as an electrode) to excite the ETM (and in some embodiments, without the addition of an electrochemical detector). In some embodiments, instead of exciting the ETM with a light or laser beam, the ETM is excited by application of a voltage and detected by an electrochemical detector, an optical detector, or both. In some embodiments, detection comprises applying an electrical voltage (and no optical stimulus) and detecting the first and second signaling moieties of a dual-labeled signal probe, wherein the first and second signaling moieties are of different modalities (e.g., ETM and OSM). In some embodiments, detection comprises adding an optical stimulus such as a laser beam or light (and no electrical voltage) and detecting the first signaling moiety and second signaling moiety wherein the first signaling moiety and the second signaling moiety are of different modalities (e.g., ETM and OSM).

A sixth aspect of the present disclosure is a method of applying an electrical voltage (and not optical stimulus) to a sample including a signal probe, where the signal probe includes only an OSM (no ETM). Said another way, an electrical voltage is applied to a sample including a signal probe, where the signal probe consists of an OSM.

A seventh aspect of the present disclosure is a method of applying an optical stimulus (such as a laser beam or light) (and no electrical voltage) to a sample including a signal probe, where the single probe includes only an ETM (no OSM). Said another way, an optical stimulus is applied to a sample including a signal probe consisting of an ETM.

An eighth of the present disclosure is directed to real-time detection of amplification. In typical real-time, PCR reactions no post PCR process is needed. However, as disclosed herein, a PCR product comprising an OSM must undergo post PCR processing, i.e., binding to a capture probe, to detect. In some embodiments, the PCR product (without an OSM) must be hybridized to a signal probe the signal probe comprising an OSM. The signal probe/amplicon complex must then be hybridized with a capture probe. In either case, energy is applied to the OSM labeled signal probe or OSM labeled amplicon to excite the optical signaling moiety which is then detected by an optical detector, electrochemical detector, or both.

A ninth aspect of the present disclosure is directed to multiplex real-time PCR. In some embodiments, a dual-labeled signal probe (including signaling moieties having different modalities) is capable of increasing the multiplexing capabilities of the assay cartridge disclosed herein. In a typical detection system, if each of 20 pads is labeled with a capture probe for a single target, then 20 targets can be detected. Here, because different optical labels can be detected with different potentials, more than one target can be detected per electrode. Further, because different optical labels can be detected with different potentials, different regions of the same target can be detected. Accordingly, one aspect of the present subject matter is directed to multiplex detection. Nucleic acid is extracted from the sample. The sample is amplified. The amplified sample is mixed with at least two signal probes, wherein the first signal probe comprises a first ETM and a first optical signaling moiety and the second signal probe comprises a first ETM and a second optical signaling moiety wherein the first optical signaling moiety and second optical signaling moiety are different and can be distinguished from one another. In some embodiments, at least two different pathogens are detected. In some embodiments, at least two different amplicons are detected (the amplicons being from the same or different pathogens).

A tenth aspect of the present disclosure is directed to SNP analysis. In particular, disclosed herein is a method for identifying a single nucleotide residue of interest at a position within a stretch of consecutive nucleotide residues in a DNA molecule. In one embodiment, a method for detecting a SNP in a target nucleic acid in a sample is provided, the method including performing an amplifying step comprising contacting the sample with a primer comprising a first nucleic acid sequence to produce an amplification product if any target nucleic acid is present in the sample; performing a hybridizing step comprising contacting the amplification product with a SNP specific signal probe comprising a second nucleic acid sequence complementary to a SNP containing region of the amplification product, the SNP specific signal probe comprising a first signaling moiety and a second signaling moiety (wherein the first signaling moiety and second signaling moiety are of different modalities); and detecting the presence or absence of the amplification product, wherein the presence of the amplification products is indicative of the presence of the SNP in the target nucleic acid target, and wherein the absence of the amplification products is indicative of the absence of the SNP in the target nucleic acid target.

An eleventh aspect of the present disclosure is directed to measuring the location of capture probes on an electrode. Instead of using the second signaling moiety to detect an analyte, the second signaling moiety is used to determine the location of the capture probe. The location of the optical signaling moiety during detection (or post manufacture) can show whether or not the capture probes are uniformly distributed across the electrode or not. In some embodiments, an optical signaling moiety is attached to the capture probe. Detecting the location/distribution of capture probes on an electrode can be used to assess or improve quality control.

A twelfth aspect of the present disclosure is a detection system comprising a single signal exciter, a first detector and a second detector wherein the first detector and second detector are of different modalities. In some embodiments, the first signal exciter is a voltage generator. In some embodiments, the first signal exciter is a light or laser beam generator. In some embodiments, the first detector is an ECM detector, and the second detector is an optical detector. In some embodiments, the system is does not include a light/laser beam source (such as lasers, photodiodes, or lamps) for activating the optical signaling label on the signal probe. In some embodiments, the system is does not include a laser, a high intensity mercury (Hg) arc lamp, a fiber optic light source, or other high intensity light source for activating the optical signaling label on an optically labeled signal probe or optically labeled nucleic acid. In some embodiments, the system is does not include a voltage generator for activating the ETM on the signal probe. In some embodiments, the system comprising a first signal exciter, a second signal exciter, a first detector, and a second detector, wherein the first signal exciter is capable of producing a voltage and the second signal exciter is capable of producing a first wavelength of light, where the first detector detects a redox/oxidation reaction, and the second detector detects a second wavelength of light.

In some embodiments, the ETM (such as an ETM included as part of a signal probe) is detected by an ETM detector, an optical detector, or both. In some embodiments, the OSM (such as an OSM included as part of a signal probe) is detected by an ETM detector, an optical detector, or both. In some embodiments, the detector is an ammeter, and it measures the plasmonic voltage from the OSM. In some embodiments, the detection instrument is designed to detect an OSM without a light or laser to excite the optical signaling moiety. In some embodiments, the detection instrument is designed to detect an optical signaling moiety without an optical detector. In some embodiments, the detection instrument is designed to detect an optical signaling moiety without a light or laser to excite the optical signaling moiety and without an optical detector. In some embodiments, the detection instrument is designed to detect an ETM without a voltage generator. In some embodiments, the detection instrument is designed to detect an ETM without an ETM detector. In some embodiments, the detection instrument is designed to detect an ETM without a voltage generator to excite the ETM and without an ETM detector.

### BRIEF DESCRIPTION OF THE DRAWINGS

FIGS. 1a and 1b show a typical sandwich assay used in electrochemical detection. In FIG. 1b, the sandwich comprises three main elements, the capture probe (2), the signal probe (3) and the target (4). The target can be synonymous with a PCR amplicon sequence in nucleic acid embodiments. The target can have a portion (4b) which binds or hybridizes specifically to a desired portion of the signal probe (3b), a portion (4a) which binds or hybridizes to a corresponding capture probe portion (2b) and optionally one or more flanking portions, e.g., (4c). The capture probe can include a linker (2a) that links, joins, or binds the capture probe (2) to the electrode surface (1). As pictured, the signal probe has a detectably labeled portion or labels (3a) that are in close proximity to the electrode surface. Herein, the circle represents an ETM, the square represents an optical signaling moiety, and the triangle represents a colorimetric moiety. The circle can represent a single ETM or a plurality of ETMs. The square can represent a single optical signaling moiety or a plurality of optical signaling moieties. The triangle can represent a single colorimetric moiety or a plurality of colorimetric signaling moieties. In some embodiments, the labeled portion 3a is conjugated or internal to the signal probe binding portion (3b). Not pictured is a self-assembled monolayer (SAM), which is also attached to the electrode surface in some embodiments via one or more linkers in similar format to the capture probe linkers (2a), and which serves to prevent or lessen undesired electron transfer events (“noise”) to the electrode surface.

FIGS. 2a-g show embodiments of the signal probes disclosed herein. In some embodiments a first signaling moiety (which is shown as 3 circles) is toward, near, or at the 5′ end of the signal probe and a second signaling moiety of a different modality than the first signaling moiety (which is shown as 3 squares) is conjugated internally to the signal probe as shown in FIG. 2a. In some embodiments, a first signaling moiety (which is shown as 1 circle but which may be a plurality of signaling moieties) is toward, near, or at the 5′ end of the signal probe and a second signaling moiety of a different modality than the first signaling moiety (which is shown as 1 square but which may be a plurality of signaling moieties) is also toward, near, or at the 5′ end of the signal probe as shown in FIG. 2b. In some embodiments, a third signaling moiety of a different modality than the first or second signaling moiety is also on the 5′ end of the signal probe as shown in FIG. 2c. In some embodiments, a first signaling moiety (which is shown as 1 circle) is toward, near, or at the 5′ end of the signal probe along with a second signaling moiety of a different modality than the first signaling moiety (which is shown as 1 square) and in some embodiments a third signaling moiety of a different modality than the first signaling moiety and second signaling moiety (which is shown as 1 triangle) is also toward, near, or at the 5′ end of the signal probe and wherein a first signaling moiety (which is shown as 1 circle) along with a second signaling moiety of a different modality than the first signaling moiety (which is shown as 1 square) and in some embodiments a third signaling moiety of a different modality (which is shown as 1 triangle) are conjugated internally to the signal probe as shown in FIG. 2d. In this example, the circle can be an ETM, the square an optical signaling moiety and the triangle a colorimetric moiety. In some embodiments, a first signaling moiety (which is shown as 3 circles) is on the 3′ end of the signal probe and a second signaling moiety of a different modality than the first signaling moiety (which is shown as 3 squares) is conjugated internally to the signal probe as shown in FIG. 2e. In some embodiments, a first signaling moiety (which is shown as 1 circle) is on the 3′ end of the signal probe and a second signaling moiety of a different modality than the first signaling moiety (which is shown as 1 square) is also on the 3′ end of the signal probe as shown in FIG. 2f. In some embodiments, a third signaling moiety of a different modality than the first or second signaling moiety is also on the 3′ end of the signal probe as shown in FIG. 2g. In some embodiments, a first signaling moiety (which is shown as 1 circle) is on the 3′ end of the signal probe along with a second signaling moiety of a different modality than the first signaling moiety (which is shown as 1 square) and in some embodiments a third signaling moiety of a different modality than the first signaling moiety and second signaling moiety (which is shown as 1 triangle) on the 3′ end of the signal probe and wherein a first signaling moiety (which is shown as 1 circle) along with a second signaling moiety of a different modality than the first signaling moiety (which is shown as 1 square) and in some embodiments a third signaling moiety of a different modality (which is shown as 1 triangle) are conjugated internally to the signal probe as shown in FIG. 2h. In this example, the circle can be an ETM, the square an optical signaling moiety and the triangle a colorimetric moiety. In some embodiments, the first signaling moiety (which is shown as 3 circles) is toward, near, or at the 5′ end of the signal probe and a second signaling moiety of a different modality than the first signaling moiety is conjugated toward, near, or at the 3′ end of the signal probe (FIG. 2i). Stated another way, the probe has two signaling moieties, which signaling moieties can be introduced toward, near, or at the 5′ terminus or anywhere in the middle of the sequence. In FIG. 2 (a-i), ii is the detection region and has one or more detection moieties, e.g., ferrocene or a fluorophore sch as fluorescein/FITC, rhodamine, Alexa Fluor, and i is the annealing region, e.g., the region that binds to the target analyte. The annealing region may or may not have one or more detection moieties. Alternatively, the signal probe may contain an extra terminal nucleoside at an end of the nucleic acid (n+1 or n+2), which are used to covalently attach the signaling moiety, but which do not participate in base pair hybridization during detection. FIG. 2j shows an extra terminal nucleoside linking the signaling moiety at the 5′ end of the signal probe and a second signaling moiety that is a different modality than the first signaling moiety is conjugated internally to the signal probe itself. FIG. 2k shows an extra terminal nucleoside linking a first signaling moiety, a second signaling moiety that is of a different modality than the first signaling moiety and a third signaling moiety that is of a different modality than the first signaling moiety and second signaling moiety at the 5′ end of the signal probe and a first signaling moiety, a second signaling moiety that is of a different modality than the first signaling moiety and a third signaling moiety that is of a different modality than the first signaling moiety and second signaling moiety conjugated internally to the signal probe itself. FIG. 2l shows an extra terminal nucleoside linking a first signaling moiety, a second signaling moiety that is of a different modality than the first signaling moiety and a third signaling moiety that is of a different modality than the first signaling moiety and second signaling moiety at the 5′ end of the signal probe. In this example, the circle can be an ETM, the square an optical signaling moiety and the triangle a colorimetric moiety. Alternatively, the primer may contain a linker which is used to covalently attach the electron transfer moiety. FIG. 2m shows a linker linking the signaling moiety at the 5′ end of the signal probe and a second signaling moiety that is a different modality than the first signaling moiety is conjugated internally to the signal probe itself. FIG. 2n shows a linker linking a first signaling moiety, a second signaling moiety that is of a different modality than the first signaling moiety and a third signaling moiety that is of a different modality than the first signaling moiety and second signaling moiety at the 5′ end of the signal probe and a first signaling moiety, a second signaling moiety that is of a different modality than the first signaling moiety and a third signaling moiety that is of a different modality than the first signaling moiety and second signaling moiety conjugated internally to the signal probe itself. FIG. 2o shows a linker linking a first signaling moiety, a second signaling moiety that is of a different modality than the first signaling moiety and a third signaling moiety that is of a different modality than the first signaling moiety and second signaling moiety at the 5′ end of the signal probe. In this example, the circle can be an ETM, the square an optical signaling moiety and the triangle a colorimetric moiety. FIG. 2p shows a linker on the 5′ end linking the first signaling moiety and 3′ end linking the second signaling moiety wherein the second signaling moiety is of a different modality than the first signaling moiety. In each embodiment in FIG. 2 (a-p) where three signaling modalities are shown, two signaling modalities could be used, i.e., just an ETM and OSM. In each embodiment in FIG. 2 (a-p) where two signaling modalities are shown, three signaling modalities could be used. In each embodiment in FIG. 2 (a-p) where three signaling modalities are shown, more than three signaling modalities could be used. In each embodiment in FIG. 2 (a-p), each circle, square or triangle can represent a single or a plurality of signaling moieties.

FIG. 3 shows an embodiment of the system disclosed herein. As seen in FIG. 3a, a capture probe bound to a detectable label (shown as a square) can be individually or serially spotted onto the detection site. As seen in FIG. 3b, the detectable label (shown as a square) can be located close to the electrode surface.

FIG. 4 shows an embodiment of the system disclosed herein. In some instances, it may be beneficial to have a longer amplicon and have multiple capture probes that bind different portions of the amplicon. The method can make use of one or more capture probes on the same detection electrode, which bind to different portions of an amplicon/target. In some embodiments, the portion of the amplicon/target (a) that binds a first capture probe (e) does not cross-hybridize with a second capture probe (d) (see FIGS. 4a and 4b). In some embodiments, the portion of the amplicon/target (b) that binds a first capture probe (d) does not cross-hybridize with a second capture probe (e) (see FIGS. 4a and 4b). In some embodiments, the second portion of the amplicon/target (b) binds a first capture probe (d) and does not cross-hybridize with a second capture probe (e) which binds a second portion of the amplicon/target (a). In some embodiments, the first portion of the amplicon/target (a) can bind a first capture probe (d) and a second capture probe (e), the second portion of the amplicon/target (b) cross-hybridizes with a second capture probe (e) and a first capture probe (d). In some embodiments, the first portion of the amplicon/target (a) can bind a first capture probe (d) and a second capture probe (e), but the second portion of the amplicon/target (b) can only bind with a second capture probe (e). As shown herein, the first signaling moiety and the second signaling moiety are both bound to the signal probe which is bound to section (c) of the amplicon. Herein, the circle represents an ETM, while the square represents a fluorescent moiety (FM). The circle and square represent a single or a plurality of signaling moieties.

FIG. 5 shows show a typical assay used herein. In FIG. 5 each signal of a plurality of probes has the same ETM but different optical signaling moieties (shown as square 1 and square 2 but could include more than 2 optical signaling moieties). In some embodiments, the first optical signaling moiety is a fluorescein and the second optical signaling moiety is a rhodamine. In some embodiments, the first optical signaling moiety is a fluorescein and the second optical signaling moiety is an Alexa Fluor 405.

FIG. 6 shows N6. N6 is a label that can be used; its synthesis is described in commonly owned U.S. Pat. No. 7,393,645, which is incorporated by reference herein in its entirety.

FIG. 7 shows QW56 and FIG. 8 shows QW80. QW56 and QW80 are ferrocene labels that can be prepared using routine DNA synthesis techniques essentially as described in commonly owned application PCT/US08/82666 (published as WO2009/061941A2 and U.S. Pat. No. 7,820,391), which are herein incorporated by reference in their entirety. In U.S. Pat. No. 9,891,215 (which is incorporated by reference herein in its entirety).

FIG. 9 shows a flow chart for a method of correlating signals from electrochemical detection and optical detection.

FIG. 10 shows an optical label, fluorophore, directly labeled to DNA, i.e., no signal probe, bound to a capture probe. Item 1 is a surface or an electrode.

FIG. 11 shows that fluorescence (produced by exciting a fluorophore by a voltage) is expected to be dependent on the concentration of the fluorophore in solution. Excitation detected by optical detector.

FIG. 12 shows that a hybridization complex can be detected by optical detection following application of a voltage to excite an optical signaling moiety. Excitation detected by optical detector.

FIG. 13 shows brightfield reflectance and autofluorescence at different emission wavelengths (blue, green, and red) of the detection electrode/gold pad.

FIG. 14 shows brightfield reflectance and far red (and a merge of the two) nucleic acid stain on the electrode.

FIG. 15 shows that specific targets, here Influenza A, can be detected on the electrode array vs negative control.

FIG. 16 shows that specific targets, here Influenza A, can be differentially detected on the electrode array where only arrays 10 and 12 have Influenza A capture probes and therefore can bind signal probe with a fluorescent marker.

FIG. 17 shows that the distribution of the capture probes can be visualized by hybridizing it to a signal probe comprising a fluorescent marker.

### DETAILED DESCRIPTION OF THE INVENTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

Unless defined otherwise, all terms of art, notations and other technical terms or terminology used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

### Overview

Disclosed herein are systems and methods for generating a detectable signal (current) by positioning a fluorophore near an electrode surface and wherein excitation of the fluorophore is caused by a voltage and the excitation of the fluorophore is detected by an optical reader, an electrochemical reader or both.

Disclosed herein are systems and methods for generating a detectable signal by positioning an ETM near an electrode surface, wherein excitation of the ETM is caused by a light/laser beam; and wherein excitation of the ETM is detected by an optical reader, an electrochemical reader, or both an optical reader and an electrochemical reader.

Disclosed herein are systems and methods for generating a detectable signal by positioning a fluorophore and ETM near an electrode surface and wherein excitation of the fluorophore and ETM is caused by a voltage or light/laser beam and the excitation of fluorophore and ETM is detected by an optical reader, an electrochemical reader, or both an optical reader and an electrochemical reader.

Disclosed herein are methods of detecting fluorescence (luminescence, chemiluminescence, phosphorescence) signatures based on excitation from an electrical signal. Normally, fluorescence or luminescence signatures are excited by light. Instead, in the embodiments disclosed herein, the electrically excited fluorophore is detected by an optical detector such as Photomultiplier tube (PMT), charge coupled device (CCD), complementary metal-oxide semiconductor (CMOS), camera, microscope, etc., and/or an electrochemical sensor such as an ammeter. In some embodiments, the electrically excited optical signaling moiety is detected by both an optical detector and an electrochemical sensor (such as an ammeter).

Disclosed herein are methods of detecting ETM (e.g., oxidation moieties, reduction moieties, redox moieties and/or transition metal complex) signatures based on excitation from a light or laser beam source. Normally, it is believed that ETM signatures are excited by current/voltage. Instead, in the embodiments disclosed herein, a redox reaction is detected by an optical detector such as PMT (Photomultiplier tube) or CCD (charge coupled device), camera, microscope, etc., and/or electrochemical sensor such as an ammeter, where the redox reaction is facilitated by irradiation of the ETM with light or a laser beam.

The notion of electrical excitation of fluorescence signaling moieties is an enormous breakthrough in fluorescence spectroscopy and its applications. The notion of optical excitation of ETMs is an enormous breakthrough in electrochemistry and its applications. The combination of electrical excitation of fluorescence signaling moieties and optical excitation of ETMs is significant.

### Definitions

As used herein, “amplification” refers to any in vitro method for increasing the number of copies of a nucleotide sequence with the use of a polymerase. Nucleic acid amplification results in the incorporation of nucleotides into a nucleic acid molecule (e.g., DNA) or primer thereby forming a new nucleic acid molecule complementary to the nucleic acid template. The formed nucleic acid molecule and its template can be used as templates to synthesize additional nucleic acid molecules. As used herein, “amplicon” refers to a nucleic acid molecule, which comprises a primer or a portion of a primer and a newly synthesized strand which is the complement of the sequence downstream of the primer binding site.

As used herein, “analyzing” refers to measuring, detecting, or determining the presence, absence, or composition of something.

As used herein, “analyte” refers to an entity that can selectively bind a capture binding ligand. Analytes may be natural, biological, or synthetic, e.g., as in any of synthetic or other molecules used for drug discovery that manifest unusually good or specific binding affinity to a “capture binding ligand.” Both analytes and capture binding ligands may consist of one or more different domains. The person of skill will appreciate that complementary orientations between the analyte and capture binding ligands are necessary. In an embodiment, the analyte may be an environmental pollutant (including pesticides, insecticides, toxins, etc.); a chemical (including solvents, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc.); whole cells (including procaryotic (such as pathogenic bacteria) and eucaryotic cells, including mammalian tumor cells); viruses (including retroviruses, herpesviruses, adenoviruses, lentiviruses, etc.); and spores; etc.

Suitable nucleic acid target analytes include, but are not limited to, the nucleic acid of any number of viruses including orthomyxoviruses, (e.g., influenza virus), paramyxoviruses (e.g. respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpesviruses (e.g., Herpes simplex virus, varicella-zoster virus, cytomegalovirus, Epstein-Barr virus), rotaviruses, Norwalk viruses, hantavirus, arenavirus, rhabdovirus (e.g., rabies virus), retroviruses (including HIV, HTLV-I and -II), papovaviruses (e.g., papillomavirus), polyomaviruses, and picomaviruses, and the like), and bacteria (including a wide variety of pathogenic and non-pathogenic prokaryotes of interest including Bacillus; Vibrio, e.g., V. cholerae; Escherichia, e.g., Enterotoxigenic E. coli, Shigella, e.g., S. dysenteriae; Salmonella, e.g., S. typhi; Mycobacterium e.g., M. tuberculosis, M. leprae; Clostridium, e.g., C. botulinum, C. tetani, C. dificile, C. perfringens; Comyebacterium, e.g., C. diphtheriae; Streptococcus, S. pyogenes, S. pneumoniae; Staphylococcus, e.g., S. aureus; Haemophilus, e.g., H. influenzae; Neisseria, e.g., N. meningitidis, N. gonorrhoeae; Yersinia, e.g., G. lamblia Y. pestis, Pseudomonas, e.g., P. aeruginosa, P. putida; Chlamydia, e.g., C. trachomatis; Bordetella, e.g., B. pertussis; Treponema, e.g., T. palladium; and the like) (collectively “Bacterial and Viral Targets”).

Suitable nucleic acid target analytes include, but are not limited to, the nucleic acid of any number of gram-positive organisms including, Bacillus cereus group, Bacillus subtilis group, Corynebacterium, Cutibacterium acnes, Propionibacterium acnes, Enterococcus, Enterococcus faecalis, Enterococcus faecium, Lactobacillus, Listeria, Listeria monocytogenes, Micrococcus, Staphylococcus, Staphylococcus aureus, Staphylococcus epidermidis, Staphylococcus lugdunensis, Streptococcus, Streptococcus agalactiae (GBS), Streptococcus anginosus group, Streptococcus pneumoniae, Streptococcus pyogenes (GAS), Resistance Genes, mecA, mecC, vanA, or vanB (collectively “Gram Positive Targets”).

Suitable nucleic acid target analytes include, but are not limited to, the nucleic acid of any number of gram-negative organisms including, Acinetobacter baumannii, Bacteroides fragilis, Citrobacter, Cronobacter sakazakii, Enterobacter (non-cloacae complex), Enterobacter cloacae complex, Escherichia coli, Fusobacterium nucleatum, Fusobacterium necrophorum, Haemophilus influenzae, Klebsiella oxytoca, Klebsiella pneumoniae group, Morganella morganii, Neisseria meningitidis, Proteus, Proteus mirabilis, Pseudomonas aeruginosa, Salmonella, Serratia, Serratia marcescens, Stenotrophomonas maltophilia, Resistance Genes, CTX-M, IMP, KPC, NDM, OXA (OXA-23 and OXA-48), or VIM (collectively “Gram Negative Targets”).

Suitable nucleic acid target analytes include, but are not limited to, the nucleic acid of any number of fungal organisms including, Candida albicans, Candida auris, Candida dubliniensis, Candida famata, Candida glabrata, Candida guilliermondii, Candida kefyr, Candida krusei, Candida lusitaniae, Candida parapsilosis, Candida tropicalis, Cryptococcus gattii, Cryptococcus neoformans, Fusarium, or Rhodotorula (collectively “Fungal Targets”).

In some embodiments, the targets are human-specific infectious disease agents or targets, with the markers or targets being nucleic acid markers. In some embodiments, the targets are human diseases, such as cancer and neurodegenerative diseases.

As used herein, “array” refers to a plurality of distinct sites bearing different capture binding ligands. In some embodiments, the array is “addressable” insofar as the individual sites have a predetermined or determinable location relative to one another, optionally with the help of electronic connectors and/or software.

As used herein, “autofluorescence” refers to fluorescence emitted naturally by a biological substance.

As used herein, a “capture binding ligand” is synonymous with a “capture probe” or “capture binding probe” and is a compound that exhibits a relatively strong or specific affinity for another compound such that it is capable of abstracting that compound away from a group of other compounds in a mixture of compounds. The capture binding ligand may be a protein, carbohydrate, nucleic acid, small molecule, or any combination of these.

As used herein, “chemiluminescence labels” refer to moieties which participate in light-producing reactions in the presence of a triggering agent (here an electrical voltage applied to an electrode) or cofactor. Examples of suitable chemiluminescence labels include but without limitation, peroxidase, bacterial luciferase, firefly luciferase, functionalized iron-porphyrin derivatives, luminal, isoluminol, acridinium esters, sulfonamide, and others. A preferred chemiluminescent label includes xanthine oxidase with hypoxanthine as substrate. The triggering agent contains perborate, a Fe-EDTA complex and luminol. Choice of the particular chemiluminescence labels depends upon several factors which include the cost of preparing labeled members, the method to be used for covalent coupling to the detector molecule, and the size of the detector molecules and/or chemiluminescence label. Correspondingly, the choice of chemiluminescence triggering agent will depend upon the particular chemiluminescence label being used.

As used herein, a “dual-labeled signal probe” comprises a first signaling moiety and a second signaling moiety, wherein the first signaling moiety and the second signaling moiety are of different modalities (e.g., one signaling moiety is an ETM while the other signaling moiety is an OSM).

As used herein, “electrode” refers to a composition, which, when connected to an electronic device, is able to sense charge or voltage and convert it to a signal. Electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo206), tungsten oxide (W03) and ruthenium oxides; and carbon (including glassy carbon electrodes, graphite, graphene, and carbon paste).

As used herein, “electrochemical detection” refers to the use of at least two electrodes to apply potential and measure current produced by a chemical reaction. “Electrochemical Detection” excludes detection of conductivity, impedance or capacitance of a droplet, a portion of a droplet, or the contents of a droplet by (i) electrochemiluminescence and (ii) by optical means.

As used herein, an “electrochemical sensor” or “electrochemical detector” or “ETM detector” refer to the detection of redox/oxidation reactions and includes potentiometric, amperometric, conductometric, and/or impedimetric sensors. “Electrochemical detectors” (and the like) exclude detectors of conductivity, impedance or capacitance of a droplet, a portion of a droplet, or the contents of a droplet by (i) electrochemiluminescence detectors and (ii) by optical detectors.

As used herein, the terms “electron donor moiety,” “electron acceptor moiety,” “electron transfer moieties” “redox-active labels” or grammatical equivalents herein refer to molecules capable of electron transfer under certain conditions. It is to be understood that electron donor and acceptor capabilities are relative; that is, a molecule which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds and selection of those compounds is within the skill of the skilled artisan. One advantage of redox-mediated electronic detection is that there is a variety of different electronic transfer moiety labels each having its own distinct potential that can be selectively measured or filtered. Some electron transfer moieties include, but are not limited to, transition metal complexes, organic electron transfer moieties, electrodes, metallocenes such as ferrocene, and ferrocene derivatives; methylene blue; and osmium. Electron transfer moieties further include oxidation moieties, reduction moieties, redox moieties and/or transition metal complex. Redox-active labels come in a variety of different potentials that can be used, similar to the existence of different color dyes and chemilumiscent compounds.

As used herein, “fluorescence” refers to a radiative transition from the lowest excited singlet state (S1) to a singlet ground state (S0) of a molecule.

As used herein, “fluorescent moieties” refer to entities which include electrons which can absorb a photon and briefly enter an excited state before either dispersing the energy non-radiatively or emitting it as a photon, but with a lower energy, i.e., at a longer wavelength (wavelength and energy are inversely proportional). The Fluorescent Moieties described herein can briefly enter an excited state after the application of an electrical voltage to the fluorescent moiety before either dispersing the energy non-radiatively or emitting it as a photon, but with a lower energy, i.e., at a longer wavelength (wavelength and energy are inversely proportional). Fluorescent moieties include fluorophores, reactive dyes, quantum dots and fluorescent proteins such as Green Fluorescent Protein (GFP), yellow fluorescent protein (YFP), blue fluorescent protein (BFP), or cyan fluorescent protein (CFP) and other optical signaling moieties as defined herein.

As used herein, “fluorophores” may include both extrinsic and intrinsic fluorophores. A fluorophore (or fluorochrome, similarly to a chromophore) is a fluorescent chemical compound that can re-emit photons upon excitation (by light or voltage/current). Fluorophores typically contain several combined aromatic groups, or planar or cyclic molecules with several z bonds. Representative fluorophores include but are not limited to Alexa Fluor® 350, Dansyl Chloride (DNS-C1), 5-(iodoacetamida)fluoroscein (5-IAF); fluoroscein 5-isothiocyanate (FITC), tetramethylrhodamine 5-(and 6-)isothiocyanate (TRITC), 6-acryloyl-2-dimethylaminonaph-thalene (acrylodan), 7-nitrobenzo-2-oxa-1,3-diazol-4-yl chloride (NBD-Cl), ethidium bromide, Lucifer Yellow, 5-carboxyrhodamine 6G hydrochloride, Lissamine rhodamine B sulfonyl chloride, Texas Red™ sulfonyl chloride, BODIPY™, naphthalamine sulfonic acids including but not limited to 1-anilinonaphthalene-8-sulfonic acid (ANS) and 6-(p-toluidinyl)naphthalene-2-sulfonic acid (TNS), Anthroyl fatty acid, DPH, Parinaric acid, TMA-DPH, Fluorenyl fatty acid, Fluorescein-phosphatidylethanolamine, Texas red-phosphatidylethanolamine, Pyrenyl-phosphatidylcholine, Fluorenyl-phosphotidylcholine, Merocyanine 540, 1-(3-sulfonatopropyl)-4-[-.beta.-[2 [(di-n-butylamino)-6 naphthyl]vinyl]pyridinium betaine (Naphtyl Styryl), 3,3′ dipropylthiadicarbocyanine (diS-C₃-(5)), 4-(p-dipentyl aminostyryl)-1-methylpyridinium (di-5-ASP), Cy-3 lodo Acetamide, Cy-5-N-Hydroxysuccinimide, Cy-7-Isothiocyanate, rhodamine 800, IR-125, Thiazole Orange, Azure B, Nile Blue, Al Phthalocyanine, Oxaxine 1, 4′, 6-diamidino-2-phenylindole (DAPI), Hoechst 33342, TOTO, Acridine Orange, Ethidium Homodimer, N(ethoxycarbonylmethyl)-6-methoxyquinolinium (MQAE), Fura-2, Calcium Green, Carboxy SNARF-6, BAPTA, coumarin, phytofluors, Coronene, green fluorescent proteins and metal-ligand complexes.

Representative intrinsic fluorophores include but are not limited to organic compounds having aromatic ring structures including but not limited to NADH, FAD, tyrosine, tryptophan, purines, pyrimidines, lipids, fatty acids, nucleic acids, nucleotides, nucleosides, amino acids, proteins, peptides, DNA, RNA, sugars, and vitamins. Additional suitable fluorophores include enzyme-cofactors; lanthanide, green fluorescent protein, yellow fluorescent protein, red fluorescent protein, or mutants and derivates thereof.

As used herein, the terms “hybridization” and “hybridizing” refer to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well-known in the art, are used.

As used herein, the term “immobilize” or derivative terms thereof, includes affixation, association, or binding, whether covalently or non-covalently.

As used herein, the term “label” refers to an entity that can signal or be stimulated to signal an event or the presence of a molecule or complex of molecules. Labels may include, e.g., dyes, radioactive atoms or molecules, redox-active compounds, enzymes, enzyme substrates, nucleic acids, derivatives thereof the like. The labels can be affixed to the detector molecule (signal probe) or capture molecule.

As used herein, “light” is used in its broad sense, meaning electromagnetic radiation, which propagates through space and includes not only visible light, but also infrared and ultraviolet radiation.

As used herein, the terms “monolayer”,” “self-assembled monolayer” or “SAM” herein is meant a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. A “mixed” monolayer comprises a heterogeneous monolayer, that is, where at least two different molecules make up the monolayer.

As used herein, the terms “multimodal” or “different modalities” refer to systems incorporating two or more modalities/techniques. Multi-modal detection can be defined as any combination of electrochemical, optical, fluorescence, surface plasmon resonance, nanoplasmonic sensors, bio-layer interferometry, chemiluminescence, spectroscopy, or colorimetric detection using two or more of any of these in combination. Signals can be produced and detected through traditional methods of each technique, or through signal generation caused by one of the other modalities and detected by a single detector. For example, a multimodal system could combine optical, electrochemical, or radioactive labels or detectors. The skilled artisan would appreciate that optical detection/signaling/imaging is a different modality as compared with electrochemical detection/ signaling/imaging.

As used herein, “optical detection” refers to the recognition of an object via the electromagnetic spectrum including, but not limited to, the visible and/or ultraviolet and/or infrared portions of the electromagnetic spectrum.

As used herein, “optical signaling molecule,” “optical signaling moieties” or “excitable molecules” refer to electromagnetic signals and can include but are not limited to, Fluorophores, Quantum Dots (Qdots); Autofluorescence; Chemiluminescence Alkaline Phosphatase and other chemiluminescence labels; Fluorospheres, i.e. fluospheres and Transfluospheres; Polymer beads doped with one or more fluorescent labels; Fluorescent Microspheres; Silicon nanoparticles; Silica and silicate doped materials; Semiconductor materials; E-type fluorescent luminophores; P-type fluorescent luminophores; Fluo-3 and Fluo-4 Calcium indicators; Calcium Green indicator; Fluozin Zinc indicators; Phen Green for the detection of a broad range of ions including Cu2+, Cu+ etc; Newport Green for the detection of Zn2+; Leadmium Green dye for the measurement of lead and cadmium; Magnesium green for the electric detection of free magnesium; Mag-fura-2 and Mag-indo-1 for magnesium detection; Mag-fluo-4 for both calcium and magnesium detection in both free solution and intercellular; Phycobiliproteins (many different forms); Bucky balls, C₆₀ etc; Carbon nanotubes; Cardio green/indocyanine green fluorescent indicators; Metallic colloids of Ag, Au, Pt, Fe Pd, Cu, Zn, Rh, Cr, Pb etc and mixed colloidal metal combinations; pH indicators such as SNARF-1, SNARF-4F, SNARF-5F, Dextran BCECF etc; 6-chloro-9-nitro-5-oxo-5H-benzo {a}phenoxazine (CNOB) for the detection of nitroreductase and nitrate reductase activity; SYTOX dead cell stains, such as SYTOX Blue, green, Orange, Red; DAPI and the Propidium Iodide labels; Probes for double stranded DNA detection such as Ethidium bromide, Picogreen and Syber green; Alexa fluorophore range of dyes; BODIPY and related structural dyes; Cellular and Organelle lights (genetically encoded proteins); Green Fluorescent Protein (GFP) and its analogues; Coumarin dyes; Prodan and related structural dyes; Voltage sensitive probes such as DisBAC₄(3) and CC2-DMPE; and/or Ncode miRNA labeling fluorophores

As used herein, “optical biosensors”, “optical sensors,” or “optical detectors” have the ability to detect light, typically at a specific range of the electromagnetic spectra (ultraviolet, visible, and infrared). The sensor detects either wavelength, frequency, or polarization of light and converts it into electric signal due to photoelectric effect. Optical detectors include fluorescence biosensors, colorimetric biosensors, Surface Enhancement Raman Scattering (SERS) biosensors, Surface Plasmon Resonance (SPR), photonic crystal based, optical resonator based, optical fiber based or optical wavelength-based sensors. Optical detectors transform optical signals into electrical ones. Optical sensors have the ability to detect light, typically at a specific range of the electromagnetic spectra (ultraviolet, visible, and infrared). The sensor detects either wavelength, frequency, or polarization of light and converts it into electric signal due to photoelectric effect. “Optical detectors” (and the like) exclude electrochemical detectors.

As used herein, “nucleic acid” or “oligonucleotide” or grammatical equivalents herein means at least two nucleotides covalently linked together. Nucleic acids generally contain phosphodiester bonds, although in some cases, as outlined below, nucleic acid analogs are included that may have alternate backbones, comprising, for example, phosphoramide. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides as well as nucleoside and nucleotide analogs, and modified nucleosides such as amino modified nucleosides. In addition, “nucleoside” includes non-naturally occurring analog structures. Thus, for example, the individual units of a peptide nucleic acid, each containing a base, are referred to herein as a nucleoside.

As used herein, the term “nucleotide” refers to a nucleoside-5′-oligophosphate compound, or structural analog of a nucleoside-5′-oligophosphate, which can act as a substrate or inhibitor of a nucleic acid polymerase. Exemplary nucleotides include, but are not limited to, nucleoside-5′-triphosphates (e.g., dATP, dCTP, dGTP, dTTP, and dUTP); nucleosides (e.g., dA, dC, dG, dT, and dU) with 5′-oligophosphate chains of 4 or more phosphates in length (e.g., 5′-tetraphosphosphate, 5′-pentaphosphosphate, 5′-hexaphosphosphate, 5′-heptaphosphosphate, 5′-octaphosphosphate); and structural analogs of nucleoside-5′-triphosphates that can have a modified base moiety (e.g., a substituted purine or pyrimidine base), a modified sugar moiety (e.g., an O-alkylated sugar), and/or a modified oligophosphate moiety (e.g., an oligophosphate comprising a thio-phosphate, a methylene, and/or other bridges between phosphates).

As used herein, the “polymerase” as used herein, refers to an enzyme that catalyzes the process of replication of nucleic acids. More specifically, DNA polymerase catalyzes the polymerization of deoxyribonucleotides alongside a DNA strand, which the DNA polymerase “reads” and uses as a template. The newly polymerized molecule is complementary to the template strand and identical to the template's partner strand. Polymerases (including DNA polymerases and RNA polymerases) include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT®) DNA polymerase, Pyrococcus furious (Pflu) DNA polymerase, DEEPVENT DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME) DNA polymerase, Methanobacterium thermoautotrophicum (Mth) DNA polymerase, Mycobacterium DNA polymerase (Mtb. Mlep), and mutants, variants, and derivatives thereof. RNA polymerases such as T3, T5 and SP6 and mutants, variants and derivatives thereof may also be used.

As used herein, the term “primer” refers to an oligonucleotide, either natural or synthetic, that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2^{nd} Edition (Cold Spring Harbor Press, New York, 2003). As used herein, “probe” refers to synthetic or biologically produced nucleic acids (DNA or RNA) which, by design or selection, contain specific nucleotide sequences that allow them to hybridize, under defined stringencies, specifically (i.e., preferentially) to target nucleic acid sequences. As with the primers, the probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length.

A primer (and/or probe) as defined herein may be chemically modified, i.e., a primer and/or probe may comprise a modified nucleotide or a non-nucleotide compound. A probe (or a primer) is then a modified oligonucleotide. “Modified nucleotides” (or “nucleotide analogs”) differ from a natural “nucleotide” by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a “nucleotide” whereby a “modified nucleotide” is obtained. A natural base in a “nucleotide” may also be replaced by, e.g., a 7-desazapurine whereby a “modified nucleotide” is obtained as well. The terms “modified nucleotide” or “nucleotide analog” are used interchangeably in the present application. A “modified nucleoside” (or “nucleoside analog”) differs from a natural nucleoside by some modification in the manner as outlined above for a “modified nucleotide” (or a “nucleotide analog”).

As used herein, a “redox-active” compound or moiety is meant one capable of transferring, shuttling, or receiving electrons from another redox-active compound or from electrodes. Some redox-active compounds include electrodes and metallocenes, including ferrocenes and derivatives thereof, methylene blue or osmium.

As used herein, the term “sample” or “sample solution” or the like refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats, and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments, or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, a “signal probe” is meant a probe molecule that bears at least one label of some sort that can bind to and signal the presence of an analyte.

As used herein, a “sensor” is an object that detects signals from its surrounding environment and converts it to meaningful or quantifiable information.

As used herein, a “solid support” or “support” refers to any material or matrix suitable for attaching oligonucleotides/capture probes. Such oligonucleotides and/or capture probes may be added or bound (covalently or non-covalently) to the supports by any technique or any combination of techniques well-known in the art. Supports may be anything other than an aqueous phase at room temperature and include, e.g., beads, gels, columns, column matrices, multi-titer plates, paper, membranes, printed circuit boards, or other array surfaces or supports.

As used herein, the terms “target analyte”, “target nucleic acid” or “target” or grammatical equivalents thereof refers to nucleic acid sequences to be amplified or detected. These include the original nucleic acid sequence to be amplified, its complementary second strand and either strand of a copy of the original sequence which is produced by replication or amplification.

The target sequence may be a portion of a gene, a regulatory sequence, genomic DNA, cDNA, RNA including mRNA and rRNA, or others. It may be any length, with the understanding that longer sequences are more specific. As will be appreciated by those in the art that the complementary target sequence may take many forms. For example, it may be contained within a larger nucleic acid sequence, i.e., all or part of a gene or mRNA, a restriction fragment of a plasmid or genomic DNA, among others. As is outlined more fully below, probes are made to hybridize to target sequences to determine the presence or absence of the target sequence in a sample. The target sequence may also be comprised of different target domains; for example, a first target domain of the sample target sequence may hybridize to a capture probe or a portion of a capture probe, a second target domain may hybridize to a portion of a different capture probe. The target domains may be adjacent or separated. The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain. A target refers to a nucleic acid molecule to which a particular primer or probe is capable of preferentially hybridizing.

As used herein, “target sequence” or “target analyte” refers to a nucleic acid sequence within the target molecules to which a particular primer or probe is capable of preferentially hybridizing.

As used herein, the term “template” as used herein refers to a double-stranded or single-stranded molecule which is to be amplified, synthesized, or sequenced.

As used herein, a “transducer” is a general device for converting energy from one form into a different form. An electrochemical sensor and optical sensor are types of transducers.

Other terms used in the fields of recombinant DNA technology and molecular and cell biology as used herein will be generally understood by one of ordinary skill in the applicable arts.

### General Description

The present disclosure provides for systems and methods for nucleic acid detection. In some embodiments, the devices include an electrochemical detector, an optical detector, or both an electrochemical detector and an optical detector. In some embodiments, the systems of the present disclosure may be equipped for electrical excitation of an optical signaling moiety but not excitation of an optical signaling moiety via light (wavelength). In other embodiments, the systems of the present disclosure may be equipped for optical (wavelength) excitation of an ETM but not excitation of an ETM via a voltage.

The present disclosure also provides for one or more reagents, such as one or more reagents suitable for detecting one or more target nucleic acids in a sample. In some embodiments, the reagents include a signal probe, such as a signal probe including an including an ETM (e.g., ferrocene, a derivative of ferrocene, or a methylene blue) and an OSM or an optical label (e.g., fluorescein).

The present disclosure also provides for methods of detecting one or more target nucleic acid molecules in a sample comprising: hybridizing a target nucleic acid molecule (e.g., DNA or a fragment of DNA) to a signal probe, such as a signal probe having an electron transfer moiety (ETM) and an optical signaling moiety (OSM) to form a signal probe-target complex. In some embodiments, the signal probe-target complex is hybridized to a capture probe attached to an electrode surface. In some embodiments, the signal probe-target complex is not hybridized to a capture probe attached to an electrode surface. In some embodiments, the method further comprises applying a voltage to the system and detecting a signal from the ETM and the OSM. In some embodiments, the voltage excites the ETM but not the OSM. In some embodiments, the voltage excites both the ETM and the OSM.

In some embodiments, the electrically excited ETM is detected by an electrochemical detector and the electrically excited OSM is detected by an electrochemical detector. In some embodiments, the electrically excited ETM is detected by an electrochemical detector and the electrically excited OSM is detected by an optical detector. In some embodiments, the electrically excited ETM is detected by an optical detector and the electrically excited OSM is detected by an optical detector. In some embodiments, a wavelength of light is applied to the system and a signal from the ETM and OSM is detected. In some embodiments, the wavelength excites the optical signaling moiety but not the electron transfer moiety. In some embodiments, the wavelength excites both the electron transfer moiety and the optical signaling moiety. In some embodiments, the light excited ETM is detected by an electrochemical detector and the light excited OSM is detected by an electrochemical detector. In some embodiments, the light excited ETM is detected by an electrochemical detector and the light excited OSM is detected by an optical detector. In some embodiments, the light excited ETM is detected by an optical detector and the light excited OSM is detected by an optical detector.

In contrast to signal probes including two signaling moieties employing the same modality, the signal probes of the present disclosure utilize use a first signaling moiety that can be detected by a first detection modality and a second signaling moiety that can be detected by a second detection modality, where the first and second detection modalities are different (e.g., electrochemical detection as the first detection modality and optical detection as the second detection modality). Applicant submits that this difference is significant because, prior to this disclosure, it was not known whether an electrochemical signal could be detected in the presence of an optical/fluorescent signal. Likewise, Applicant submits that it was not known whether an optical/fluorescent signal could be detected in the presence of an electrochemical signal. Applicant further submits that it was also not known whether an optical/fluorescent signal would inhibit an electrochemical signal or visa-versa. The present disclosure provides for a multiplexed clinical diagnostic device which utilizes a combination of two different modalities, and which have been found to be complementary to each other.

Some embodiments of the present disclosure do not use two excitation methods. In some embodiments, a single excitation modality, e.g., electricity or light, is used to excite both the ETM and OSM. In some embodiments, while a single excitation method is used, two or more different detection modalities are used: a first detection modality (such as one having a first detector) for the first signaling moiety (e.g., an ETM) and a second detection modality (such as an one having a second detector, such as an optical detector) for the second, optical signaling moiety. In some embodiments, a single excitation method is used, and a single detection modality is used (e.g., a first detector for the ETM and optical signaling moiety).

In some embodiments, two excitation methods are utilized: electricity is used to excite the ETM, and light is used to excite the FM. In some embodiments, two excitation methods are used, and two or more detection modalities are used: a first detector for the ETM and a second optical detector for the second, optical signaling moiety. In some embodiments, two excitation methods are used, and a single detection modality is used: a first detector for the ETM and optical signaling moiety.

In some embodiments, the optical label is detected directly from the PCR product (i.e., not hybridized to a signal probe or capture probe) and the ETM is then detected via hybridization to a capture probe and application of an electric voltage though the electrode.

When developing assays which utilize a sandwich assay (capture probe bound to an amplicon bound to a signal probe) it can be difficult to find portions of the target organism sequence that are highly conserved (i.e., detectable across many variants) but also uniquely identifies the target organism of interest. This is made even more complicated when the highly conserved but unique identifier must be long enough to allow both capture and signal probe binding wherein the signal probe has not one but two signaling moieties.

### General Background on the Value of Nucleic Acid Testing

Labeling probes with ferrocene has provided a relatively sensitive means for facilitating detection of probe hybridization. Several patents address electrochemical detection of nucleic acids, for example U.S. Pat. No. 10,001,476 (which is incorporated by reference herein in its entirety) discloses detection with capture probe controls, and various capture and signal probe configurations and combinations of configurations that can facilitate accurate and efficient multiplex analyte detection. U.S. Pat. No. 10,001,476 discloses single stranded DNA on an electrode (capture probe) that binds to a target. Such systems require that the ferrocene label be held in close proximity to the electrode to work. In order to achieve the proper orientation of the ferrocene label to the electrode, sandwich assays have been used. As discussed above, sandwich assays require an amplicon capable of binding to both a capture and signal probe. Developing probes capable of binding both a capture and signal probe is complicated especially when the probe must hold not one but two signaling moieties of different modalities in the proper orientation and distance from the electrode to be excited and detected by their respective detectors. In multiplex reactions, the system must further be able to distinguish two signals of the same modality from one another (and other signals of different modalities on the pad).

### Electrochemical Detection

The systems and methods of the present disclosure include forming one or more hybridization complexes on detection electrodes under conditions where a signal probe binds to target nucleic acid. The hybridization complex of the signal probe and the target nucleic acid is then able to bind to a capture probe and hold the two different signaling moieties in close proximity to a detection electrode for excitation and detection. In some embodiments, the signal probe comprises two different signaling moieties, where each of the two different signaling moieties have different detection modalities (“dual-labeled signal probe”). In some embodiments, the two different signaling moieties can be excited using a single detection method and detected using two different detectors having different modalities or, in some embodiments, a single detector. In some embodiments, a first signaling moiety of the two different signaling moieties is an ETM; and a second signaling moiety of the two different signaling moieties is an optical label or OSM.

In some embodiments, the dual-labeled signal probe can be electrically excited via application of a voltage to produce a first signal at a detectable wavelength and a second signal that transfers electrons to the electrode. In some embodiments, the dual-labeled signal probe can be optically excited via application of (i.e., irradiated with) a light or laser beam to produce a first signal at a detectable wavelength and a second signal that transfers electrons to the electrode. In some embodiments, the dual-labeled signal probes, which are not bound to the capture probe diffuse away from the electrode when the voltage/current is turned on and are not detected. In some embodiments, the signal probe generates a measurable current when a first amperometric potential is applied.

In some embodiments, when multiple capture probes are used, each is specific for a different portion of the common nucleic acid sequence of interest. In some embodiments, when multiple capture probes are used, each is specific for a different target analyte of interest. In some embodiments, the nucleic acid sequence of interest can be non-amplified or amplified nucleic acid, e.g., through PCR. In some embodiments, the electrodes have a self-assembling monolayer (“SAM”). In some embodiments, the electrodes have a mixed SAM of two or more species, each species featuring different chain lengths, conjugated bond numbers (if any) and/or substituents (if any).

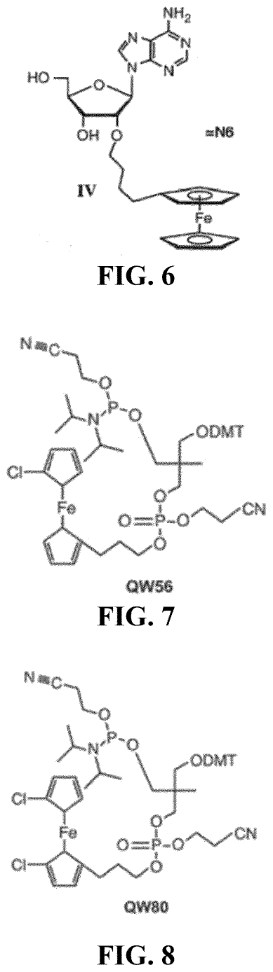

In some embodiments, a first signaling moiety of the dual-labeled signal probe is a ferrocene or a derivative of ferrocene. In some embodiments, the ferrocene or a derivative of ferrocene is selected from N6, QW56 and/or QW80. See FIG. 6, FIG. 7, and FIG. 8. In some embodiments, the ferrocene or a derivative of ferrocene is detected electrochemically, optically, or both electrochemically and optically. In some embodiments, an optical label of the dual-labeled signal probe may be detected electrochemically, optically, or both and optically.

In some embodiments, a capture oligonucleotide is immobilized on a gold surface. In some embodiments, the capture oligonucleotide is immobilized on a carbon-based electrode such as graphite, graphene, soft and hard carbon, or nanocarbons. In other embodiments, the capture oligonucleotide is immobilized on an electrode. In some embodiments, the electrodes include an insulating self-assembled monolayer or mixed monolayer.

In some embodiments, a capture oligonucleotide is immobilized to a substrate. In some embodiments, the substrate comprises a printed circuit board (PCB). Thus, in general, the suitable substrates include, but are not limited to, fiberglass, teflon, ceramics, glass, silicon, mica, plastic (including acrylics, polystyrene and copolymers of styrene and other materials, polypropylene, polyethylene, polybutylene, polycarbonate, polyurethanes, Teflon™, and derivatives thereof, etc.), GETEK (a blend of polypropylene oxide and fiberglass), etc.

In some embodiments, the detection chamber and electrode are part of a cartridge that can be placed into a device comprising electronic components (an AC/DC voltage source, an ammeter, a processor, a read-out display, temperature controller, light source, fluorescence detector, etc.). In this embodiment, the interconnections from each electrode are positioned such that upon insertion of the cartridge into the device, connections between the electrodes and the electronic components are established.

Techniques for electrochemical detection are known to those of ordinary skill in the art. Generally, at least a first input signal (AC/DC voltage) is applied to the assay complex and an output signal (such as in nanoamps, uA, mA etc) is received. The output signal is then processed to detect the presence of target analytes. Some embodiments utilize a plurality of assay hybridization complexes each attached to a different cell or pad of a detection array. This basic mechanism is described in U.S. Pat. Nos. 5,591,578, 5,770,369, 5,705,348, and PCT US97/20014 (which are all herein incorporated by reference).

### Optical Detection

Techniques for optical detection are known to those of ordinary skill in the art. Generally, at least a first input signal (light or laser beam) is applied to the assay complex and an output signal (fluorescence) is received. The output signal is then processed to detect the presence of said target analytes. A conventional fluorescent analyzer includes an optical unit for irradiating light onto a sample and a detection unit for detecting light emitted from the sample. The optical unit generally includes a light source, a dichroic mirror, an objective lens, and a sample holder. The detection unit includes an optical detector, for example, a photo multiplier tube (PMT), and a filter which transmits a specific wavelength of light, etc. The light source may include various sources, such as a halogen lamp, a light-emitting diode (LED), a laser, etc. Light emitted from the light source is reflected by the dichroic mirror and is partially absorbed by the sample. The light emitted from the sample is transmitted through the dichroic mirror and enters the detection unit. The light, which enters the detection unit is transmitted through the filter, thus having a specific wavelength and the optical detector detects the intensity of light having the specific wavelength. By analyzing the intensity of the fluorescence light emitted from the sample by varying the wavelength property of the filter or the light source, the analyte of the sample can be identified.

### Nucleic Acid Testing With a Single-Use Device

An exemplary method of nucleic acid testing is described herein. Although the various elements (steps) are discussed as sequential steps having a prescribed order, it should be understood that the process is exemplary and not intended to be limiting. Persons of ordinary skill will recognize that many of the various elements (steps) can be performed in different orders than described herein, can be performed simultaneously or substantially simultaneously with other elements (steps), or can be omitted altogether. Thus, the order of the elements (steps) discussed is not limiting.
Step 1: load sample.
Step 2: extract DNA.
Step 3: combine DNA with amplification reagents.
Step 4: amplify DNA to produce a double stranded amplicon.
Step 5: incubate the double stranded amplicon with exonuclease to form a single stranded amplicon.
Step 6: combine the single stranded amplicon with a signal probe wherein the signal probe has a first signaling moiety and a second signaling moiety wherein the first signaling moiety and a second signaling moiety are of different modalities, i.e., electrochemical and optical to form a signal probe-amplicon complex.
Step 7: combine the signal probe-amplicon complex with a capture probe.
Step 8: excite the first signaling moiety and the second signaling moiety with a voltage (AC/DC voltage).
Step 9: detect, by electro-sensor detection, the first signaling moiety; and detect, by optical detection, the second signaling moiety.
Step 10: correlate the detected electro-sensor signal and detected optical signal.
Step 11: produce a detection result based on the correlated electro-sensor signal and optical signal.

In some embodiments, before the electro-sensor signal and optical signal are correlated, the electro-sensor signal and/or optical signal are processed by removing a subset of signals or filtering the signals. In some embodiments, the detection result is based on the correlated signal, the electro-sensor signal, and the optical signal. In some embodiments, the detection result is based only on the correlated signal. In some embodiments, the detection result is based on the correlated signal and the electro-sensor signal or the optical signal.

In some embodiments, the excitation of the first signaling moiety and the second signaling moiety with a voltage comprises applying a first voltage to excite the first signaling moiety and applying second voltage to excite the second signaling moiety. In some embodiments, the excitation of the first signaling moiety and a second signaling moiety with a voltage comprises applying a first voltage to excite the first signaling moiety and the second signaling moiety.

In some embodiments, the capture probe comprises a third signaling moiety. In some embodiments, the capture probe comprises a third signaling moiety, where the third signaling moiety is of the same type (i.e., detection modality) as the second signaling moiety, e.g., both the second and third signaling moieties are optical signaling moieties.

In some embodiments, a first signal probe comprises two different signaling moieties having different detection modalities (e.g., an ETM and an OSM); and where a second signal probe comprises two different signaling moieties having different detection modalities (e.g., an ETM and an OSM); where signals from at least one of the two different signaling modalities of the first signal probe can be distinguished from signals from at least one of the two different signaling modalities of the second signal probe (e.g., two different OSMs may be distinguishable from each other).

In some embodiments, the first signaling moiety is a ferrocene tag; and the second signaling moiety is an optical tag; and where the first and second detectable moieties undergo a detectable change in one or more properties upon application of a voltage (or upon application of a light). Non-limiting examples of the one or more properties that may change are signal intensity, electrochemical potentials, and/or reaction constants.

In some embodiments, detection occurs without amplification, i.e., steps 3 and 4 are omitted. In some embodiments, detection occurs without a signal probe, i.e., fluorescently labeled nucleic acid binds directly to a capture probe, i.e., step 6 is omitted. In some embodiments, the signal probe only has an OSM (no ETM) which is excited by application of a voltage. In some embodiments, the signal probe only has an OSM (no ETM) and the OSM signal is detected by an electrochemical detector. In some embodiments, the signal probe only has an ETM (no OSM) which is excited by application of light or laser beam. In some embodiments, the signal probe only has an ETM (no OSM) and the ETM signal is detected by an OSM detector. In some embodiments, Step 9, detecting by electro-sensor detection detects the first signaling moiety and the second signaling moiety (there is no optical detection). In some embodiments, Step 9, detecting by optical detection detects the first signaling moiety and the second signaling moiety (there is no electrochemical detection).

### PCR Generally

Conventional PCR techniques are disclosed in U.S. Pat. Nos. 4,683,202; 4,683,195; 4,800,159; and 4,965,188, the disclosures of which are hereby incorporated by reference herein in their entireties. U.S. Pat. Nos. 5,210,015; 5,487,972; 5,804,375; 5,804,375; 6,214,979; and 7,141,377 disclose real-time PCR and TaqMan® techniques, the disclosures of which are hereby incorporated by reference herein in their entireties.

The polymerase chain reaction (PCR) is a relatively simple technique that amplifies a DNA template to produce specific DNA fragments in vitro. A typical amplification reaction includes target DNA, a thermostable DNA polymerase, two oligonucleotide primers (5′ and 3′), deoxynucleotide triphosphates (dNTPs), reaction buffer and magnesium chloride. Each cycle of PCR includes steps for template denaturation, primer annealing and primer extension. The initial step denatures the target DNA by heating it. In the denaturation process, the two intertwined strands of DNA separate from one another, producing the necessary single-stranded DNA template for replication by the thermostable DNA polymerase. In the next step of a cycle, the temperature is reduced so that the oligonucleotide primers can form stable associations (anneal) with the denatured target DNA and serve as primers for the DNA polymerase. Finally, the synthesis of new DNA begins. An enzyme called “Taq polymerase” synthesizes-builds-two new strands of DNA, using the original strands as templates. This process results in the duplication of the original DNA, with each of the new molecules containing one old and one new strand of DNA. Then each of these strands can be used to create two new copies, and so on, and so on. The cycle of denaturing and synthesizing new DNA is repeated as many as 30 or 40 times, leading to more than one billion exact copies of the original DNA segment. The cycling process of PCR is typically automated in a thermocycler, which is programmed to alter the temperature of the reaction to allow DNA denaturing and synthesis.

The amplicon disclosed herein may be produced in any amplification reaction including PCR, 5-RACE, Anchor PCR, “one-sided PCR,” LCR, NASBA, SDA, RT-PCR, real-time PCR, quantitative PCR, quantitative RT-PCR, and other amplification systems known in the art. In some embodiments, the reaction is run at a single temperature (isothermal). These isothermal methods include the cycling probe reaction, strand displacement, Invader™, SNPase, rolling circle reaction and NASBA.

In some embodiments, in the absence of nucleic acid synthesis, there should be little or no electron transfer between the signal probe and the electrode. In some embodiments, the system is sensitive enough that amplification is not needed in order to bind the target analyte to the signal probe and detect it.

### Förster Resonance Energy Transfer (FRET)

FRET technology (see, for example, U.S. Pat. Nos. 4,996,143, 5,565,322, 5,849,489, and 6,162,603 each of which is incorporated by reference herein in their entireties) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength.

In one example, an oligonucleotide probe can contain a donor fluorescent moiety and a corresponding quencher, wherein the donor FM is excited by a voltage and dissipates the transferred energy in a form other than light or dissipates the transferred energy in the form of a wavelength. When the probe is intact, energy transfer typically occurs between the two fluorescent moieties such that fluorescent emission from the donor fluorescent moiety is quenched. During an extension step of a polymerase chain reaction, a probe bound to an amplification product is cleaved by the 5′ to 3′ exonuclease activity of, e.g., a Taq polymerase such that the fluorescent emission of the donor fluorescent moiety is no longer quenched. Exemplary probes for this purpose are described in, e.g., U.S. Pat. Nos. 5,210,015; 5,994,056; and 6,171,785 each of which is incorporated by reference. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Calif.), Iowa Black™, (Integrated DNA Tech., Inc., Coralville, Iowa), BlackBerry™ Quencher 650 (BBQ-650), (Berry & Assoc., Dexter, Mich.).

Fluorescent analysis can be carried out using, for example, a photon counting epifluorescent microscope system (containing the appropriate dichroic mirror and filters for monitoring fluorescent emission at the particular range), a photon counting photomultiplier system, or a fluorometer. Excitation to initiate energy transfer can be carried out with an Argon ion laser, a high intensity mercury (Hg) arc lamp, a fiber optic light source, or other high intensity light source appropriately filtered for excitation in the desired range.

Fluorescent donor and corresponding acceptor moieties are generally chosen for (a) high efficiency Förster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothiocyanatostilbene-2,2′-disulfonic acid, 7-diethylamino-3-(4′-isothiocyanatophenyl)-4-methylcoumarin, succinimdyl 1-pyrene-butyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm for the purpose of the present disclosure is the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640-NHS-ester, can be combined with C6-Phosphoramidites (available from ABI (Foster City, Calif.) or Glen Research (Sterling, Va.)) to produce, for example, LC Red 640-Phosphoramidite. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3′-amino-CPGs that require coupling of a fluorescein-NHS-ester after oligonucleotide synthesis.

FRET can also cause a reduction in the plasmonic electricity.

Disclosed is a method of detecting DNA on an electrode surface using voltage to excite a fluorescent donor. In some embodiments, the excitation of the fluorescent donor is detected by an optical detector, an electrochemical detector or both.

Disclosed is signaling oligonucleotide wherein the oligonucleotide comprises an ETM, a fluorescent donor and a fluorescent accepter.

Disclosed is a signal probe/target/capture probe hybridization complex, which upon excitation of the ETM and OSM on the signal probe via application of a voltage, energy is transferred to the electrode surface and a current is induced. In some embodiments, the current is a plasmonic current. In some embodiments, the plasmonic current is a direct measure of fluorescence, phosphorescence or chemiluminescence signatures. In some embodiments, the plasmonic current is a direct measure of target analyte in a sample, i.e., the direct measure of a hybridization event.

### Probe Synthesis, Functionalization and Conjugation—Generally Design of Synthetic Oligonucleotides

Regarding the design of synthetic oligonucleotides for use in amplification reactions, Rychlik et al., (1989, Nucleic Acids Research, vol 17(21):8543-8551) and Rychlik (1995, Molecular Biotechnology, vol 3: 129-134) (both incorporated by reference in their entireties), describe selection criteria and computer programs to design probes and primers. Both teach that probes should not generate secondary structure or exhibit self-hybridization. U.S. Pat. No. 6,495,323 (incorporated by reference in its entirety) describes in detail the formation of probes attached to an electron transfer moiety.

### Capture Probe Synthesis

U.S. Pat. No. 10,001,476 discloses in more detail probe synthesis, functionalization and conjugation and its disclosure is incorporated by reference in its entirety. Probe synthesis, functionalization and conjugation are all well-known techniques in the art.

Nucleic acid capture probes are typically designed to be complementary to a roughly 40- to 50-base sequence within the target. The capture probe sequence is usually complementary to the 3′-region of the target (but the reverse-5′-can also be true), and is designed to have a melting temperature (TM) of ~50 °C. Capture probes can be modified either at the 3′ end or the 5′ end with a disulfide linker for covalent attachment to a gold electrode surface, e.g., as essentially described in commonly owned U.S. Pat. Nos. 6,753,143 and 7,820,391, each of which is herein incorporated by reference in their entireties.

Capture probes, including, e.g., nucleic acids, can be adhered to electrodes or other substrate surfaces directly or indirectly, covalently, or noncovalently, using a variety of well-known techniques. See, e.g., Ch. 13, Chemically Modified Electrodes, Martin and Foss, pp. 403-442, Laboratory Techniques in Electroanalytical Chemistry; 2d Ed., Kissinger and Heineman, Eds., MARCEL DEKKER, INC. (1996); Biochip Technology, Cheng and Kricka, Eds. George H. Buchanan Printing Company, Bridgeport, N.J. (2001) (both incorporated by reference in their entireties).

### Signal Probe Synthesis

The signal probe sequence(s) is/are complementary to specific region(s) of the target and not to any region(s) of the capture probe. The signal probe sequence is usually complementary to the 5′-region of the target (but the reverse—3′—can also be true). If sequence discrimination is needed, the sequence polymorphism should be as close as possible to the center of the amplicon sequences, and the TMs of the two amplicons should be as closely matched as possible. Ferrocene labels (typically 1-6 labels per probe) are added toward, near, or at the 5′-terminus of the signal probe sequence(s). Optical labels (typically 1-10 labels per probe) are added toward, near, or at the 5′-terminus and/or a different location of the signal probe sequence(s). Since hybridization must take place at a single temperature, the TM values of all signal probes should be within a range of 5 °C. Since all detection reactions must occur within the same solution, signal probes and capture probes must be designed to avoid any cross-hybridization; maximum AGo values for cross-hybridization have been empirically established.

In some embodiments, one ferrocene or a derivative of ferrocene is added toward, near, or at the 5′-terminus of the signal probe sequence and one optical signaling label is added next to the ferrocene or a derivative of ferrocene toward, near, or at the 5′-terminus of the signal probe sequence. In some embodiments, two ferrocene labels are added toward, near, or at the 5′-terminus of the signal probe sequence and two optical signaling labels are added toward, near, or at the 5′-terminus of the signal probe sequence. In some embodiments, 1-10 ferrocene labels are added three ferrocene labels are added to the 5′-terminus of the signal probe sequence the 5′-terminus of the signal probe sequence and 1-10 optical signaling labels are added three ferrocene labels are added to the 5′-terminus of the signal probe sequence the 5′-terminus of the signal probe sequence. In some embodiments, a plurality of ferrocene labels is added toward, near, or at the 5′-terminus of the signal probe sequence and a plurality of optical signaling labels are added toward, near, or at the 5′-terminus of the signal probe sequence.

In one embodiment, a nucleic acid is modified with at least two detectable labels at one location i.e., next to each other. In one embodiment, a nucleic acid is modified with at least two detectable labels at two locations, i.e., not next to each other. In one embodiment, a nucleic acid is modified with more than two detectable labels at more than two locations, i.e., not next to each other. In one embodiment, a nucleic acid is modified with a plurality of detectable labels at a plurality of locations (next to each other or not). For example, to increase the signal obtained from the probe, a plurality of detectable labels at a plurality of locations may be used. For example, the detectable labels can be attached both 5′ and 3′ or anywhere in between. In one embodiment, the plurality of detectable labels is the same, to result in a uniform signal. Alternatively, each of the plurality of detectable labels may be different (a different modality such as electrochemical, optical, radioisotope, etc.).

In some embodiments the detectable labels on the signal probe are held 1 base pair away from the capture probe and still produces a detectable signal. In some embodiments the detectable labels on the signal probe are held anywhere from 1-10 base pairs away from the capture probe and still produces a detectable signal. In some embodiments the detectable labels on the signal probe are held anywhere from 1-50 base pairs away from the capture probe and still produces a detectable signal. In some embodiments the detectable labels on the signal probe are held anywhere from 1-100 base pairs away from the capture probe and still produces a detectable signal. In some embodiments the detectable labels on the signal probe are held anywhere from 36-72 base pairs away from the capture probe and still produces a detectable signal.

In some embodiments, the ETM is positioned such that the gap between the ETM and optical label (e.g., OSM) is zero bases. In some embodiments, the sequence gap between the ETM and optical label is zero to two bases. In some embodiments, an ETM label is between about 5 and about 50 base pairs away from an optical label, such as between about 5 and 100 base pairs away from an optical label, or such as between about 5 and 500 base pairs away from an optical label.

In some embodiments, ferrocene, a derivative of ferrocene, or methylene blue signaling moiety of a signal probe is greater than 5 base pairs away from the optical label, such as greater than 10 base pairs away, such as greater than 50 base pairs away, such as greater than 100 base pairs away, such as greater than 200 base pairs away from the optical label.

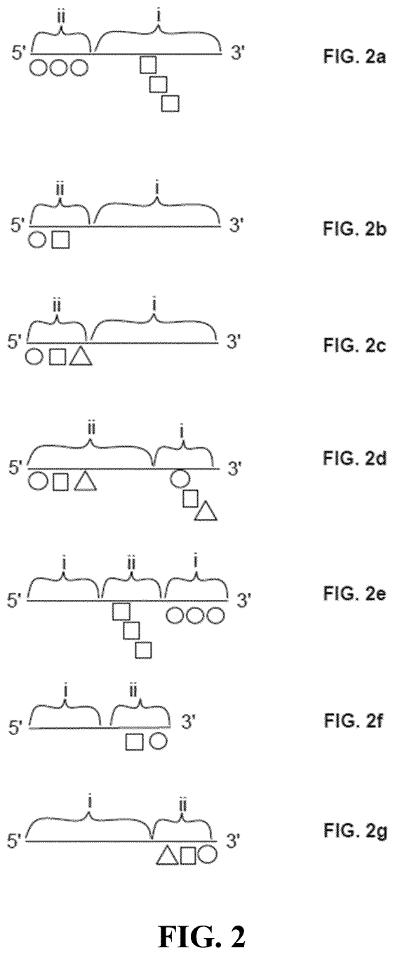

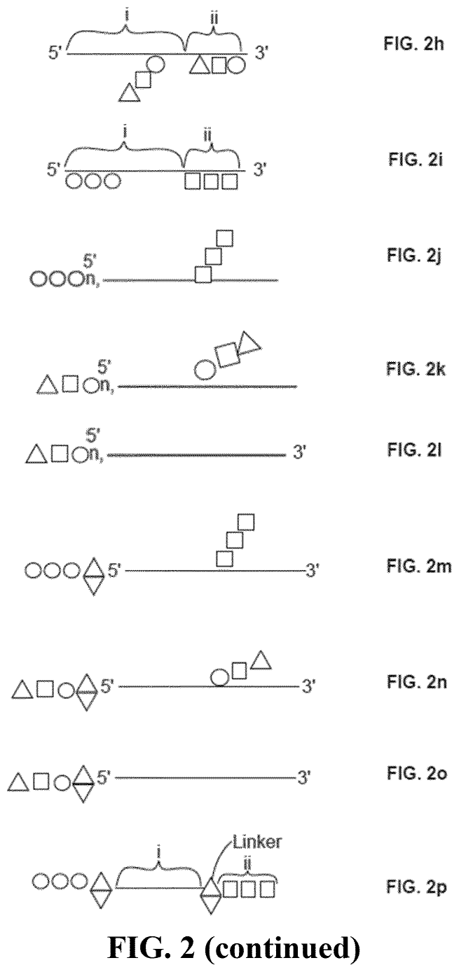

When synthesizing the signal probe, the electron transfer moiety and the optical label can be covalently attached to the nucleic acid in a variety of positions: the 5′ end and the middle of the signal probe (FIG. 2a), the 3′ end and the middle of the sequence (FIG. 2e), or at both the 5′ and 3′ ends (FIG. 2d). In an embodiment, the attachment is via attachment to the base of the nucleoside, or via attachment to the backbone of the nucleic acid, including either to a ribose of the ribose-phosphate backbone or to a phosphate moiety. In embodiments, the compositions are designed such that the electron transfer moieties are as close to the “π-way” as possible. Attachment of the ferrocene tag and optical signaling label should not perturb annealing of the amplicon to the signal probe.

Alternatively, the signal probe may contain an extra terminal nucleoside at an end of the nucleic acid (n+1 or n+2), which are used to covalently attach the electron transfer moieties or optical labels but which do not participate in base pair hybridization to the amplicon similar to what is shown in FIG. 2j and FIG. 2l where the circles represent an ETM, squares represent an optical moiety and triangles represent a colorimetric moiety.

Alternatively, it may be desirable to insert a linker arm that separates the electron transfer moiety from the probe-amplicon binding region. FIG. 2m-2p where the circles represent an ETM, squares represent an optical moiety and triangles represent a colorimetric moiety.

The signal probes are synthesized using standard phosphoramidite chemistry and can include any nucleotide or modified base, which is amenable to DNA binding. The nucleic acid portion of the signal probes can be DNA or RNA or chimeric mixtures or derivatives or modified versions thereof. In addition to being labeled with an electrochemically detectable label and optical label, the signal probes can be modified at the base moiety, sugar moiety, or phosphate backbone, and may include other appending groups or labels.

The signal probes can be any suitable size but ideally do not bind to the capture probe. In some embodiments, the signal probes are in the range of 10-30 nucleotides or 20-30 nucleotides, or 15-30 nucleotides, or 10-25 nucleotides although signal probes may be longer or shorter depending upon the need.

In some embodiments, the signal probes are devoid of enhancing groups. In some embodiments, the signal probes do not undergo a detectable change in any observable property upon hybridization to the amplicon or upon hybridization to the capture probe. In some embodiments, the signal probes only undergo a detectable change upon application of a voltage. In some embodiments, the signal probes only undergo a detectable change upon excitation with a light or laser beam. In some embodiments, the signal probes undergo a detectable change upon application of a voltage and excitation with a light or laser beam.

The signal probes can be labeled (as described above) using any known labeling method. As an example, the signal probes may be labeled by: (1) attachment at the sulfur of a phosphorothioate linkage; (2) attachment at a 2′-amino group; (3) attachment at the 1′ position using an appropriately modified sugar containing an alkylamine substituted carboxamide, for example; (4) attachment at the 1′ position using an basic site, for example, and an alkyl diamine as a linker, for example; (5) creation of a structure by reductive alkylation of the adduct formed between an alkyl diamine and an abasic site; (6) incorporation using 4′-thio-2′-deoxyuridine or 4′-thiothymidine; (7) attachment at the 2′-position of 4-thiothymidine or 4-thio-2′-deoxyuridine; (8) attachment at the 4-amino position of deoxycytidine, if the 4-amino group is derivatized with an alkylamine; (9) attachment through the 6′ position of adenine, if the 6-amino group is derivatized with an alkylamino moiety; (10) incorporation using the 8′ position of adenine if this position is substituted with an alkyl thioamine; (11) attachment at the N2 of guanine, if the N2 amino is derivatized with an alkylamino group; or (12) attachment at the N2 position of aminoadenine if the 2-amino group is derivatized with an alkylamine.

All signal probes may be purified using techniques known in the art.

### False Positive Results

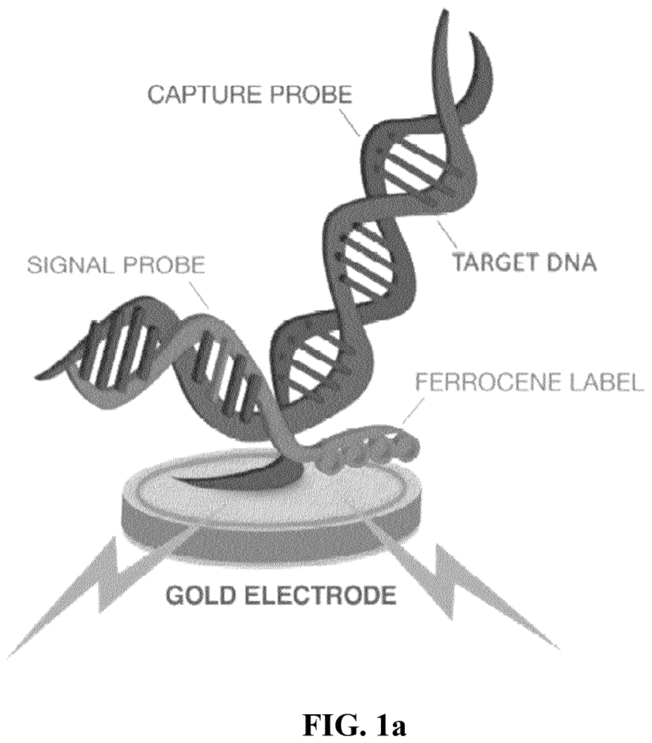

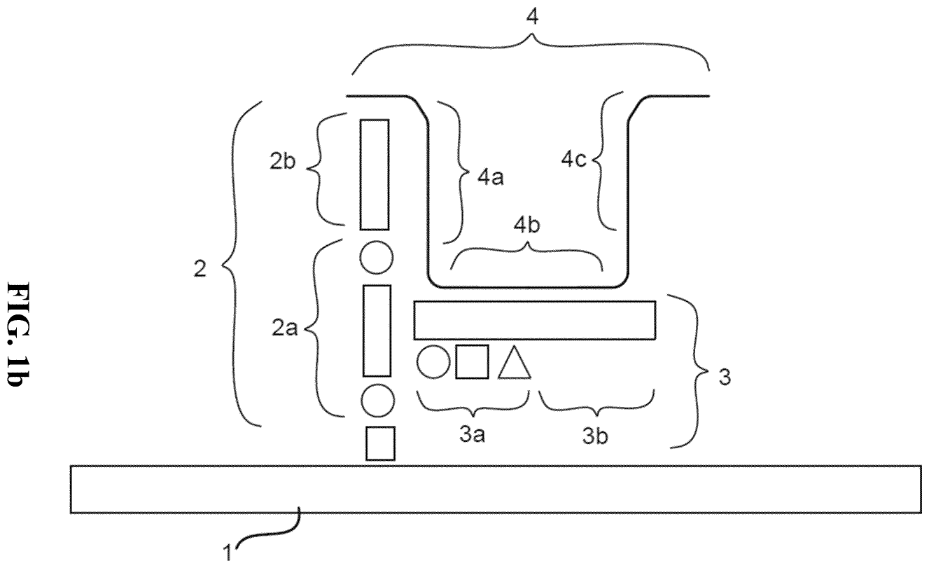

False positive results can cause serious problems including the unnecessary use of antibiotics, antimicrobials, or antifungal treatments. To avoid false positive results the signal probe should not bind to the capture probe. For example, in FIG. 1b section 3b should not bind to the capture probe (only the amplicon), thus reducing false positives.

### False Negative Results

False negative results are rarer. In prior art systems, it is not known whether a negative signal was the result of no amplicon, or failure of the signal probe/capture probe to form a sandwich. By having a second label on the signal probe, false negatives can be reduced.

### Compositions

### Dual-Labeled Signal Probe

Disclosed herein are signal probes, such as signal probes including a first signaling moiety and a second signaling moiety, such as where the first and second signaling moieties are detectable using different detection modalities. In some embodiments, the first signaling moiety is an ETM. In some embodiments, the second signaling moiety is one of an OSM or an FM.

Also disclosed herein are compositions comprising a first signal probe comprising a first signaling moiety and a second signaling moiety. In some embodiments, the first and second signaling moieties are detectable using a different detection modality. In some embodiments, the first signaling moiety is detected by a first transducer and the second signaling moiety is detected by a second transducer, wherein the first transducer and second transducer are of a different modality. In some embodiments, the first signal probe may include a third signaling moiety. In some embodiments, the compositions further comprise a second signal probe, which is different than the first signal probe (e.g., a second signal probe which includes at least one different signaling moiety as compared with the first signal probe).

Suitable signaling moieties include any detectable labels, including, but not limited to, labels detectable optically or electrochemically. Suitable signaling moieties include any molecules that can be detected via luminescence, fluorescently, chemiluminescence, phosphorescence, bioluminescence, electronically, electrochemically, radioactively, electrochemiluminescence, enzymes, Förster-resonance energy transfer (FRET), Surface Plasmon Resonance (SPR), photonic crystal based, optical resonator based, optical fiber based, optical wavelength based, and/or via RAMAN technologies. In some embodiments, signaling moieties include, but are not limited to, electron transfer moieties, fluorescent moieties, radioisotopic moieties, optical dyes, RAMAN labels, etc.

In some embodiments, the first signaling moiety is an ETM; and the second signaling moiety is an OSM. In some embodiments, the first signaling moiety comprises ferrocene; and the second signaling moiety is an OSM. In some embodiments, the first signaling moiety comprises methylene blue; and the second signaling moiety is an OSM.

In some embodiments, the first signaling moiety is an ETM; and the second signaling moiety is a fluorescent moiety (FM). In some embodiments, the first signaling moiety comprises ferrocene; and the second signaling moiety is a fluorescent moiety. In some embodiments, the first signaling moiety comprises methylene blue; and the second signaling moiety is a fluorescent moiety. In some embodiments, the fluorescent moiety is fluorescein. In some embodiments, the ETM and fluorescent moiety are excited by a voltage and the fluorophore is induces a mirror dipole in the metallic material causing plasmonic current flow.

In some embodiments, the signal probe comprises a third signaling moiety, such as a corresponding acceptor fluorescent moiety, e.g., a quencher. In some embodiments, the signal probe may comprise a third signaling moiety whose signal is distinguishable from another OSM of the signal probe. By way of example, the signal probe may include three signaling moieties, where two of the signaling moieties are OSMs, and where each of the two OSMs are different. In some embodiments, the probe may include at least one ETM, at least one FM, and at least one quencher moiety. In some embodiments, the signal probe includes only an optical label; and the optical label is excited by application of a voltage. In some embodiments, the signal probe has only an ETM and the ETM is excited by application of a light or laser beam.

### Dual-Labeled Signal Probe-Nucleic Acid Complex

Disclosed are compositions comprising a signal probe comprising a first signaling moiety and a second signaling moiety wherein the first signaling moiety and second signaling moiety are of a different detection modality and wherein the signal probe is bound to a target nucleic acid. In some embodiments, the first signaling moiety is detected by a first transducer and the second signaling moiety is detected by a second transducer, wherein the first transducer and second transducer are of a different modality. In some embodiments, the first signaling moiety is a first electron transfer moiety and a second signaling moiety is an optical signaling moiety. In some embodiments, the second signaling moiety is a fluorescent moiety. In some embodiments, the first signaling moiety is a ferrocene or osmium label and the second signaling moiety is a fluorescent moiety. Disclosed are compositions comprising a signal probe comprising at least one ETM and at least one FM wherein the signal probe is bound to a target nucleic acid.

### Capture Probe-Signal Probe-Nucleic Acid Complex

Disclosed are compositions comprising a first signal probe comprising a first signaling moiety and a second signaling moiety wherein the signal probe is bound to a target nucleic acid (“signal probe-nucleic acid complex”) and wherein the signal probe-nucleic acid complex is bound to a capture probe. In some embodiments, the first signaling moiety is detected by a first transducer and the second signaling moiety is detected by a second transducer, wherein the first transducer and second transducer are of a different modality. Disclosed are compositions comprising a signal probe comprising at least one ETM and at least one FM wherein the signal probe is bound to a target nucleic acid and wherein the signal probe-nucleic acid complex is bound to a capture probe.

In some embodiments, the first signal probe comprises an ETM and an OSM, wherein the signal probe is bound to a target nucleic acid and wherein the signal probe-nucleic acid complex is bound to a capture probe. In some embodiments, the first signal probe comprises a ETM and an FM, wherein the signal probe is bound to a target nucleic acid and wherein the signal probe-nucleic acid complex is bound to a capture probe. In some embodiments, the first signal probe comprises ferrocene or a derivative of ferrocene and a fluorescent moiety. In some embodiments, the first signal probe comprises methylene blue and a fluorescent moiety.

In some embodiments, the first signal probe comprises an ETM and a fluorescein label. In some embodiments, the ETM is ferrocene or a derivative of ferrocene. In some embodiments, the ETM is methylene blue.

### Capture Probe-Signal Probe-Nucleic Acid System Complex

Disclosed are compositions comprising a signal probe comprising a first signaling moiety and a second signaling moiety wherein the signal probe is bound to a target nucleic acid (“signal probe-nucleic acid complex”), wherein the signal probe-nucleic acid complex is bound to a capture probe (“capture probe-signal probe-nucleic acid complex”) wherein the capture probe-signal probe-nucleic acid complex is bound to an electrode (“capture probe-signal probe-nucleic acid system complex”). In some embodiments, the electrode comprises a self-assembled monolayer (SAM). In some embodiments, the electrode is a gold electrode, silver electrode, or platinum electrode.

The hybridization complexes described herein can be more fully understood by reference to the following numbered paragraphs:
Paragraph 1. A hybridization complex comprising a capture probe bound to an amplicon, the amplicon bound to a signal probe, the signal probe comprising nucleic acids, a first detectable label and a second detectable label wherein the first detectable label and a second detectable label are of a different modality.
Paragraph 2. A hybridization complex comprising a capture probe bound to an amplicon, the amplicon bound to a signal probe the signal probe comprising an electron transfer moiety, and an optical signaling moiety or a radioactive moiety.
Paragraph 3. A hybridization complex comprising a capture probe bound to an amplicon, the amplicon bound to a signal probe the signal probe comprising (i) one of ferrocene, a ferrocene derivative, methylene blue, or osmium; and (ii) fluorescein or a radioactive moiety.
Paragraph 4. A hybridization complex comprising a capture probe bound to an amplicon, the amplicon bound to a signal probe, the signal probe comprising nucleic acids, an electron transfer moiety and an optical signaling moiety.
Paragraph 5. A hybridization complex comprising a capture probe bound to an amplicon, the amplicon bound to a signal probe, the signal probe comprising nucleic acids, an electron transfer moiety and an optical signaling moiety wherein the amplicon comprises a first portion and a second portion and the capture probe is capable of binding the first portion but not the second portion.
Paragraph 6. A hybridization complex comprising a capture probe bound to an amplicon, the amplicon bound to a signal probe, the signal probe comprising nucleic acids, an electron transfer moiety and an optical signaling moiety wherein the amplicon comprises a first portion and a second portion and the capture probe is capable of binding the second portion but not the first portion.
Paragraph 7. A hybridization complex comprising a capture probe bound to an amplicon, the amplicon bound to a signal probe, the signal probe comprising nucleic acids, an electron transfer moiety and an optical signaling moiety wherein the amplicon comprises a first portion and a second portion and the capture probe is capable of binding the second portion and the first portion.
Paragraph 8. A hybridization complex comprising a capture probe bound to an amplicon, the amplicon bound to a signal probe, the signal probe comprising nucleic acids, an electron transfer moiety and an optical signaling moiety wherein the hybridization complex is bound to an electrode, the electrode comprising a monolayer.

### Signal Probe for Multiplex Detection

Disclosed are compositions comprising a first signal probe comprising a first electron transfer moiety and a second signaling moiety; and a second signal probe comprising the first electron transfer moiety and a third signaling moiety, wherein the first electron transfer moiety is of a different modality than the second signaling moiety, and wherein the second signaling moiety and third signaling moiety are of the same modality but the signal produced by the second signaling moiety and third signaling moiety are distinguishable. In some embodiments, second and third signaling moieties are optical signaling moieties. In some embodiments, second and third signaling moieties are fluorescent signaling moieties. In some embodiments, the first signaling moiety is ferrocene or a derivative of ferrocene and the second and third signaling moieties are fluorescent signaling moieties. In some embodiments, the first signaling moiety is a methylene blue and the second and third signaling moieties are fluorescent signaling moieties. In some embodiments, at least one of the second or third signaling moieties is fluorescein. In some embodiments, the second signaling moiety is fluorescein and third signaling moiety is a rhodamine label. Disclosed are compositions comprising a first signal probe comprising ferrocene or a derivative of ferrocene and fluorescein and a second signal probe comprising ferrocene or a derivative of ferrocene and an Alexa Fluor 405 label.

Disclosed are compositions comprising a first signal probe comprising at least one ETM and a FM wherein the signal probe is bound to a first portion of target nucleic acid and wherein the first signal probe-nucleic acid complex is bound to a capture probe; and a second signal probe comprising at least one ETM and a FM, where the FM signal of the first signal probe is distinguishable from the FM signal of the second signal probe, wherein the second signal probe is bound to a second portion of target nucleic acid and wherein the second signal probe-nucleic acid complex is bound to a capture probe.

In some embodiments, the first signaling moiety is detected by a first transducer and the second signaling moiety and third signaling moiety are detected by a second transducer, wherein the first transducer and second transducer are of a different modality.

### Fluorescently Stained Nucleic Acid on an Electrode

Disclosed are compositions comprising fluorescently stained nucleic acid bound to a capture probe wherein the Capture probe is bound to an electrode.

In some embodiments, a fluorescently stained nucleic acid is transported to an electrode where it is bound by a capture probe to form a capture probe/stained DNA hybridization complex. In some embodiments, a voltage is applied to the capture probe/stained DNA hybridization complex. In some embodiments, the fluorescently stained nucleic acid produces a detectable signal in response to the applied voltage. In some embodiments, the detectable signal is detected by an optical detector. In some embodiments, the detectable signal is detected by an electrochemical detector. In this way, fluorescently stained nucleic acid is measured by application of a voltage. In some embodiments the detectable signal is a dipole moment, plasmonic current flow, and/or fluorescing.

### Excitable Probes or Labels

Generally, a traditional fluorophore may be used as an excitable molecule source that emits energy that is detectable by an optical detector. Disclosed are excitable probes or labels that will produce a detectible signal by an optical detector when the probes or labels are excited by an electrical voltage. Disclosed are fluorophores excited by an electrical voltage that emit energy to induce a mirror dipole moment in a metallic surface. Applicant discovered that excitable probes or label sources will produce plasmonic electricity when the probes or labels are in the near field, i.e., close-to the metal structures, and excited by a voltage. In some embodiments, the FM produces a measurable plasmonic current flow which can be detected by a current flow detector. In some embodiments, the FM produces a measurable plasmonic current flow which can be detected by an optical detector.

Disclosed are excitable probes or labels including, but are not limited to, Quantum Dots (Qdots); Chemiluminescence Alkaline Phosphatase and other chemiluminescence labels; Fluorospheres, i.e. fluospheres and Transfluospheres; Polymer beads doped with one or more fluorescent labels; Fluorescent Microspheres; Silicon nanoparticles; Silica and silicate doped materials; Semiconductor materials; E-type fluorescent luminophores; P-type fluorescent luminophores; Fluo-3 and Fluo-4 Calcium indicators; Calcium Green indicator; Fluozin Zinc indicators; Phen Green for the detection of a broad range of ions including Cu²⁺, Cu⁺ etc; Newport Green for the detection of Zn²⁺; Leadmium Green dye for the measurement of lead and cadmium; Magnesium green for the electric detection of free magnesium; Mag-fura-2 and Mag-indo-1 for magnesium detection; Mag-fluo-4 for both calcium and magnesium detection in both free solution and intercellular; Phycobiliproteins (many different forms); Bucky balls, C₆₀ etc; Carbon nanotubes; Cardio green/indocyanine green fluorescent indicators; Metallic colloids of Ag, Au, Pt, Fe Pd, Cu, Zn, Rh, Cr, Pb etc and mixed colloidal metal combinations; pH indicators such as SNARF-1, SNARF-4F, SNARF-5F, Dextran BCECF etc; 6-chloro-9-nitro-5-oxo-5H-benzo {a}phenoxazine (CNOB) for the detection of nitroreductase and nitrate reductase activity; SYTOX dead cell stains, such as SYTOX Blue, green, Orange, Red; DAPI and the Propidium Iodide labels; Probes for double stranded DNA detection such as Ethidium bromide, Picogreen and SYBR green; Alexa fluorophore range of dyes; BODIPY and related structural dyes; Cellular and Organelle lights (genetically encoded proteins); Green Fluorescent Protein (GFP) and its analogues; Coumarin dyes; Prodan and related structural dyes; Voltage sensitive probes such as DisBAC₄(3) and CC2-DMPE; and/or Ncode miRNA labeling fluorophores.

Fluorophores belong to several common chemical classes including coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, OR, TheroFisher Scientific, 11^{th} Edition. In other embodiments, the fluorophore is selected from xanthene derivatives, cyanine derivatives, squaraine derivatives, naphthalene derivatives, coumarin derivatives, oxadiazole derivatives, anthracene derivatives, pyrene derivatives, oxazine derivatives, acridine derivatives, arylmethine derivatives, and tetrapyrrole derivatives. In other embodiments, the fluorescent moiety is selected from a CF dye (available from Biotium), DRAQ and CYTRAK probes (available from BioStatus), BODIPY (available from Invitrogen), ALEXA FLUOR (available from Invitrogen), DYLIGHT FLUOR (e.g. DYLIGHT 649) (available from Thermo Scientific, Pierce), Atto and Tracy (available from Sigma Aldrich), FLUOPROBES (available from Interchim), ABBERIOR Dyes (available from Abberior), DY and MEGASTOKES Dyes (available from Dyomics), SULFO CY dyes (available from Cyandye), HIL YTE FLUOR (available from AnaSpec), SETA, SETAU and SQUARE Dyes (available from SETA BioMedicals), QUASAR and CAL FLUOR dyes (available from Biosearch Technologies), SURELIGHT Dyes (available from APC, RPEPerCP, Phycobilisomes) (Columbia Biosciences), and APC, APCXL, RPE, BPE (available from Phyco-Biotech, Greensea, Prozyme, Flogen). Other non-limiting examples of fluorescent dyes include FAM (5- or 6-carboxyfluorescein), VIC, NED, PET, Fluorescein, FITC, IRD-700/800, CY3, CY5, CY3.5, CY5.5, CY7, HEX, TET, TAMRA, JOE, ROX, BODIPY TMR, Oregon Green, Rhodamine Green, Rhodamine Red, Texas Red, or Yakima Yellow.

A molecule that is capable of fluorescing includes, but is not limited to fluorophores, chromophores, lumophores, biomolecules or any molecule or device that provides for intrinsic or extrinsic luminescence activity.

In one aspect, disclosed are bioassay systems comprising electrodes for the enhancement of effects of chemiluminescence based reactions positioned near the electrode surface, wherein excitable probes or labels are excited by a voltage applied to the electrode that can be measured with an optical detector, electrochemical detector, or both. In one aspect, disclosed are bioassay systems comprising electrodes for the enhancement of effects of electrochemical based reactions using ETMs positioned near the electrode surface, wherein excitable ETM probes or labels are excited by a light or laser beam that can be measured with an optical detector, electrochemical detector, or both.

In some embodiments, the electrical excitation may also include the use of microwave energy or sonic energy to increase any reaction rates in an assay detection system. In some embodiments, the light or laser beam stimulation may also include the use of microwave energy or sonic energy to increase any reaction rates in an assay detection system.

In some embodiments, the assay systems of the present disclosure do not comprise a light or laser beam source for directing an energy beam on any included fluorophore. In some embodiments, the assay systems of the present disclosure include comprise a light or laser beam source for directing an energy beam on any included fluorophore to provide excitation energy. In some embodiments, the laser beam may be positioned adjacent to the system for directing the beam at the molecular components. In some embodiments, the laser may be any device capable of focusing an energy beam at a particular point on the solid or liquid source material for excitation and the laser may transmit RF, infrared, microwave to UV energy. Any source, known to one skilled in the art may be used, such as a laser that emits light, wherein light is used in its broad sense, meaning electromagnetic radiation which propagates through space and includes not only visible light, but also infrared and ultraviolet radiation. Thus, a single instrument placed above the surface of the assay can be used to generate the energy to excite fluorescing molecules. The light can be emitted from a fiber continuously or intermittently, as desired. Further, 2-photon excitation may be used at approximately 375 to 900 nm using continuous or short pulse width (<50 ps), high repetition rate (>1 MHz), laser diode sources. A variety of pulsed laser diode sources that will be compatible with fluorophores can be used with the present invention and are commercially available.

In some embodiments, the fluorescence caused by a voltage may be observed at distances according to the type of excitable molecule to be detected. For example, induction of fluorescence may be observed when a fluorophore is positioned from about 5 nm to about 200 nm to the electrode surface. In some embodiments, distances are about 5 nm to about 50 nm, and in some embodiments, 10 nm to about 30 nm to the electrode surface. At this scale, there are few phenomena that provide opportunities for new levels of sensing, manipulation, and control. In addition, devices at this scale may lead to dramatically enhanced performance, sensitivity, and reliability with dramatically decreased size, weight, and therefore cost.

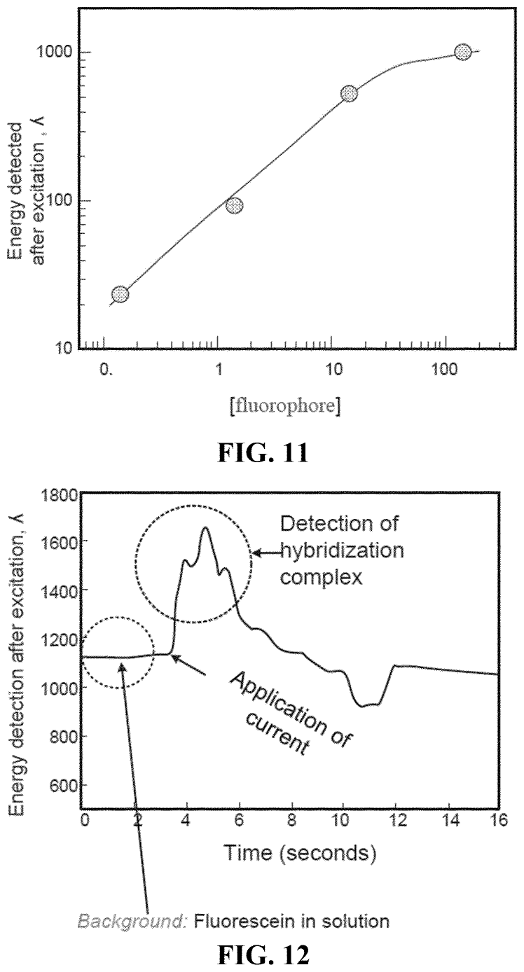

Applicant has discovered that a fluorophore can be excited by the application of current and/or voltage to produce fluorescence. Interestingly, the greater the concentration of fluorophore present, there is a corresponding increase in induced signal. In some embodiments, the more fluorophore present the more excitation voltage is needed. FIG. 11, shows the expected extent of energy-induced fluorescence on the concentration of fluorescein (a fluorescent probe) in water. It is expected that the fluorescence increases significantly over the 3 log₁₀ concentrations of fluorescent probe studied. This result suggests that the more fluorophore present close to the electrode, then the greater the induced fluorescence. It is interesting to note, that in Traditional Fluorescence-based immunoassays, the extent of detected fluorophore (usually fluorescence intensity) is directly related to the analyte concentration to be determined in the assay. Thus, it is expected that fluorescence-based immunoassays can be constructed on electrode surfaces, where the concentration of analyte (antigen) can be determined by the voltage-induced fluorescence. Remarkably, even though the excitation is purely digital (voltage), fluorescence is read indirectly (optically). The signal can be converted and displayed digitally. In contrast, fluorescence-based immunoassays in existence today, are not excited digitally (i.e., with an applied voltage). As such, the present approach is a significant breakthrough in how fluorescence is generated, measured, and quantified. In some embodiments, the concentration of the target is based on or is correlated with the plasmonic current.

### Signal Probes

During detection, signal probes are not directly bound to the electrode but bind the amplicon/capture probe, which holds them in place for detection.

In some embodiments, the signal probe is comprised of deoxyribonucleic acid, ribonucleic acid, peptide nucleic acid, PEG-modified nucleic acid, hexa-polyethylene glycol modified nucleic acid, chimeric mixtures or derivatives or modified versions thereof.

In some embodiments, the signal probe has no overlap with the capture probe. In some embodiments, the signal probe overlaps with the capture probe by about 1-10 base pairs.

A signal probe is shown in FIG. 2(d-g), where ii is the detection moiety region, e.g., a region which includes the one or more signaling moieties (e.g., ETM/optical signaling moiety), and i is the annealing region, e.g., the region that binds to the amplicon detection. In some embodiments, the signal probe has two regions: a first region capable of binding to the amplicon FIG. 2a item I, and a second region capable of producing a signal as described in 2a item ii. In some embodiments, the signal probe comprises three regions: a first region capable of binding to the amplicon FIG. 2p item i, a second region capable of producing a signal during redox mediated electronic detection FIG. 2p item ii, and a region third linking the first and second regions. See FIG. 2l (linker symbolized as a diamond).

The signal probes described herein can be more fully understood by reference to the following numbered paragraphs:
Paragraph 1. A signal probe comprising a nucleic acid, an electron transfer moiety and an optical signaling moiety, the nucleic acid comprising a first portion and a second portion wherein the first portion is capable of binding to a first capture probe but not a second capture probe and wherein the second portion is capable of binding to the second capture probe but not the first capture probe.
Paragraph 2. A signal probe comprising a nucleic acid, an electron transfer moiety and an optical signaling moiety, the nucleic acid comprising a first portion and a second portion wherein the first portion is capable of binding to a first capture probe and a second capture probe, and wherein the second portion is capable of binding to a first capture probe but not a second capture probe.
Paragraph 3. A signal probe comprising a nucleic acid, an electron transfer moiety and an optical signaling moiety, the nucleic acid comprising a first portion and a second portion wherein the first portion is capable of binding to a first capture probe and a second capture probe and wherein the second portion is capable of binding to a first capture probe and a second capture probe.
Paragraph 4. A signal probe described in paragraphs 1-3 wherein the electron transfer moiety is attached toward, near, or at the 5′ end of the signal probe and wherein the optical signaling moiety is attached toward, near, or at the 5′ end of the signal probe.
Paragraph 5. A signal probe comprising a capture probe binding region, a target analyte binding region, an electrochemically detectable label region and an optically detectable label region.

The signal probes disclosed herein may be still better understood by the following numbered paragraphs.
Paragraph 1. A probe comprising a nucleic acid, a first detectable label and a second detectable label wherein the first detectable label and second detectable label are different modalities.
Paragraph 2. A probe comprising a nucleic acid, a first electron transfer moiety and a second detectable label, which is not a traditional electron transfer moiety.
Paragraph 2a. A probe comprising nucleic acid, a first signaling moiety and an optical signaling moiety wherein the first signaling moiety is not a traditional optical signaling moiety.
Paragraph 3. A probe comprising a nucleic acid, ferrocene or a derivative of ferrocene and a second detectable label, which is not a ferrocene label.
Paragraph 3a. A probe comprising a nucleic acids, methylene blue, and a second detectable label which is not a ferrocene label.
Paragraph 3b. A probe comprising a nucleic acid, a first signaling moiety and a fluorophore wherein the first signaling moiety is not a fluorophore.
Paragraph 4. A probe comprising a nucleic acid, an electron transfer moiety wherein the electron transfer moiety is attached toward, near, or at the 5′ end of the probe and a second optical label attached toward, near, or at the 3′ end of the probe.
Paragraph 5. A probe comprising a nucleic acid, a first electron transfer moiety capable of producing a signal in response to an application of a first voltage and a second label capable of producing an optical signal in response to the application of the first voltage wherein the second label is not an electron transfer moiety.
Paragraph 5a. A probe comprising a nucleic acid, a first electron transfer moiety capable of producing an electrochemical signal in response to the application of light or a laser beam and a second label capable of producing an optical signal in response to the application of the light or a laser beam.
Paragraph 5b. A probe comprising a nucleic acid, a first electron transfer moiety (e.g., ferrocene or methylene blue) capable of producing an optical signal in response to the application of light or a laser beam and a second label capable of producing an optical signal in response to the application of the light or a laser beam wherein the first label is not an optical signaling moiety.
Paragraph 6. A probe comprising a nucleic acid, an electrochemically detectable label and an optically detectable label.
Paragraph 7. A probe comprising a nucleic acid, an electron transfer moiety (e.g., ferrocene or methylene blue) wherein the electron transfer moiety is attached toward, near, or at the 5′ end of the probe and a second optical label attached toward, near, or at the middle of the probe sequence.
Paragraph 8. A probe comprising a nucleic acid, a first electron transfer moiety (e.g., ferrocene or methylene blue) and a second detectable label which is not an electron transfer moiety wherein the first electron transfer moiety and the second detectable label are both capable of being detected after application of a voltage to the probe.
Paragraph 9. A probe comprising a nucleic acid, a first electron transfer moiety (e.g., ferrocene or methylene blue) and a second detectable label which is not an electron transfer moiety wherein the first electron transfer moiety is capable of being detected after application of a first voltage to the probe and the second detectable label is capable of being detected after application of a second voltage to the probe wherein the first voltage and second voltage are different.
Paragraph 10. A probe comprising a nucleic acid, a first electron transfer moiety (e.g., ferrocene or methylene blue) and a second detectable label which is not an electron transfer moiety wherein the first electron transfer moiety and the second detectable label are both capable of being detected after application of a voltage to the probe wherein the first electron transfer moiety is detected by an electrochemical sensor and the second detectable label is detected by a sensor which is not an electrochemical sensor.

### Detection Systems

### Integrated Nucleic Acid Test Cartridge

Integrated multiplex target analysis systems are known and are described in U.S. Pat. No. 10,864,522, the disclosure of which is hereby incorporated by reference in its entirety.

Integrated nucleic acid test cartridges capable of performing amplification and detection are disclosed. Generally, the integrated nucleic acid test cartridges are capable of receiving a sample, extracting DNA, combining DNA with amplification reagents, amplifying DNA, incubating amplicon with exonuclease, combining signal probes (wherein the signal probe comprises a ETM and optical signaling moiety) with a target, combining the signal probe/target with a capture probe to form a hybridization complex. In some embodiments, a voltage is applied to the hybridization complex and electrosensor detection of the ETM and optical detection of the OSM is performed based on the applied voltage. In some embodiments, a voltage is applied to the hybridization complex and optical detection of both signaling moieties is performed based on the applied voltage. In some embodiments, a voltage is applied to the hybridization complex and electrosensor detection of both signaling moieties is performed based on the applied voltage. In some embodiments, a light or laser beam is applied to the hybridization complex and electrosensor detection of the ETM and optical detection of the OSM is performed based on the applied light. In some embodiments, a light or laser beam is applied to the hybridization complex and optical detection of both signaling moieties is performed based on the applied light. In some embodiments, a light or laser beam is applied to the hybridization complex and electrosensor detection of both signaling moieties is performed based on the applied light.

In some embodiments, disclosed herein is an integrated cartridge for nucleic acid testing that operates in conjunction with at least one reader instrument. Disclosed herein is an integrated cartridge for nucleic acid testing that operates in conjunction with a first reader instrument and a second reader instrument wherein the first reader instrument and second reader instrument are of a different modality. In some embodiments, the first reader instrument reads a first signaling moiety wherein the first reader instrument and first signaling moiety are of the same modality and a second reader instrument reads a second signaling moiety wherein the second reader instrument and second signaling moiety are of the same modality and wherein the first reader instrument and second reader instrument are of a different modality and wherein the first signaling moiety and second signaling moiety are of a different modality.

In some embodiments, disclosed herein is an integrated cartridge for nucleic acid testing that operates in conjunction with only one reader instrument to detect a signal from two signaling moieties of different modalities. In some embodiments, the reader instrument is a ETM reader, and it reads a signal from an ETM and an OSM. In some embodiments, the reader instrument is an optical reader, and it reads a signal from an ETM and an OSM.

### Imaging Optical Labels Following Excitation From an Electrical Voltage

Disclosed is a detection system comprising:
a. one or more capture probes on an electrode surface bound to at least one fluorophore, wherein the fluorophore is capable of producing a detectable signal when a voltage is applied to the electrode;
b. an amperometric energy source to excite the fluorophore;
c. a detector.

In some embodiments, the fluorophore is positioned from about 5 nm to about 50 nm from the one or more capture probes. In some embodiments, the detector is an optical detector, an electrochemical detector or both.

The present invention includes fluorescence, luminescence, chemiluminescence or phosphorescence components that have the ability to emit light energy when contacted with a photon in the range from UV to IR. In some embodiments, the detectable signal of the excitable probe is fluorescence.

### Imaging ETM Labels Following Excitation From a Light or Laser Beam Source

Disclosed is a detection system comprising:
a. (e.g., ferrocene or methylene blue) capture probes on an electrode surface bound to at least one ETM wherein the ETM is capable of producing a detectable signal when a light or laser beam is applied to the ETM;
b. a light or laser beam source to excite the ETM;
c. a detector.

In some embodiments, the ETM is positioned from about 5 nm to about 50 nm from the one or more capture probes. In some embodiments, the detector is an optical detector, an electrochemical detector or both.

In some embodiments, the electrodes are separated by a sufficient distance to provide optimal signal readings, wherein the separation is from about from about 5 nm to 100 nm.

### Imaging Fluorescently Stained Nucleic Acid on an Electrode

Nucleic acids can be fluorescently labeled via binding, intercalation, or covalent modification. In general, a covalent fluorescent label can either be introduced directly into the nucleic acid of interest, or in a two-step approach, meaning that first a reactive handle is installed that allows for subsequent post-synthetic functionalization using the above mentioned click reactions. Direct chemical labeling via solid-phase synthesis, two-step chemical labeling via solid-phase synthesis, direct chemo-enzymatic labeling, two-step chemo-enzymatic labeling are all known techniques to label nucleic acids. See Chem Soc Rev. 2020 December 7; 49(23): 8749-8773 which is incorporated herein in its entirety. In recent years, ribozymes and deoxyribozymes have become versatile tools to label RNA with fluorescent tags, either directly or in two steps.

In some embodiments, fluorescently stained nucleic acid is transported to an electrode where it is bound by a capture probe to form a capture probe/stained DNA hybridization complex. A current/voltage is applied to the capture probe/stained DNA hybridization complex. The fluorescently stained nucleic acid produces a detectable signal in response to the applied voltage.

In some embodiments, the detectable signal is detected by an optical detector. In this way, fluorescently stained nucleic acid is measured by application of a voltage. In some embodiments, the detectable signal is detected by an electrochemical detector. FIG. 10 shows a fluorescently labeled (F) nucleic acid (A-C) bound to capture probe (e) which is bound to an electrode (1). The fluorophore is excited upon application of energy to the electrode to produce a signal detectable by an optical detector, electrochemical detector, or both.

### System for Generating Electrical Current

Disclosed are systems for generating electrical current, the system comprising:
i. a substrate;
ii. a set of electrically conductive electrodes communicatively coupled to the substrate; and
iii. an excitable probe that emits fluorescence, luminescence, or phosphorescence signatures when excited by electromagnetic energy and such excitation induces a signal detectable by an optical detector or both an optical detector and electrochemical detector.

Importantly, the current is increased as the number of excitable probes increases, thereby providing for an assay that provides an electrical signal proportional to the amount of binding of excitable probes to target substances. Stated another way, the electrical signal is proportional to the amount of binding of signal-target hybridization complex to capture probes. FIG. 12 shows that a hybridization complex is expected to be detected by optical detection following application of a voltage to excite the optical signaling moiety.

The method and system described above may be used in multiple detecting systems, including but not limited to, immunoassays, hybridization assays, resonance energy transfer assays, polarization/anisotropy-based assays, chemiluminescence based assays, luminescence-based assays, enzyme-linked immunosorbent assays.

### System for Measuring Chemiluminescence

Disclosed is a system for measuring chemiluminescence, the system comprising:
i. a capture molecule having an affinity for a desired molecule, the capture molecule positioned on a surface substrate, wherein the substrate is an electrode or connected to a set of electrodes;
ii. a detector molecule having an affinity for the desired molecule, wherein the detector molecule comprises a chemiluminescence label;
iii. a triggering component that reacts with the chemiluminescence label to generate a chemically induced electronically exited state when bound to the desired molecule and capture molecule, wherein the chemically induced electronically exited state is measured by an optical detection device and such chemically induced electronically exited state is proportional to the amount of desired molecule in the testing sample.

In some embodiments, the capture molecule may include biotin, which binds to avidin or streptavidin. In other embodiments, the capture molecule may include a thiolated molecule which binds to gold particles. In yet other embodiments, the capture molecule may include an amine-terminated molecule to bind to an NHS-activated molecule. In some embodiments, the capture molecule includes immobilized antibodies, which may be used to bind to molecules including or conjugated to specific antigenic molecules. In other embodiments, the capture molecule includes an antigenic molecule, which may be used to bind to an immobilized antibodies. In some embodiments, the capture molecule includes enzymes, which may be used to bind to molecules including or conjugated to specific enzyme substrates. In other embodiments, the capture molecule includes a substrate for an enzyme, which may be used to bind to an enzyme. In some embodiments, the capture molecule includes receptors, which may be used to bind to molecules including or conjugated to specific receptor ligands. In other embodiments, the capture molecule includes one or more receptor ligands, which may be used to bind to molecules including receptors. In some embodiments, the capture molecule includes lectins, which may be used to bind to molecules including or conjugated to specific polysaccharides. In other embodiments, the capture molecule includes one or more polysaccharides, which may be used to bind to molecules including or conjugated to one or more lectins. In even further embodiments, the capture molecule includes one or more nucleic acid sequences which may be used to bind to molecules including or conjugated to complementary base sequences. In other embodiments, the capture molecule may include tethered DNA/RNA aptamers, which may specifically bind to target analytes such as small molecules, peptides, proteins, cells.

In some embodiments, the chemically induced electronically exited state is able to generate photons in the range of UV to IR.

Still further, disclosed is using the present concept of electronically exciting a fluorophore using a voltage and measuring the excited state of the fluorophore with an optical detector including a microscope that can provide visual images but also measuring the induced plasmonic current flow. In some embodiments, the excitable probes or labels generate metallic surface plasmons and induce a mirror dipole in the metallic structures and generate a current flow in the solution, which is detected.

### Correlating Electrochemical and Optical Signals

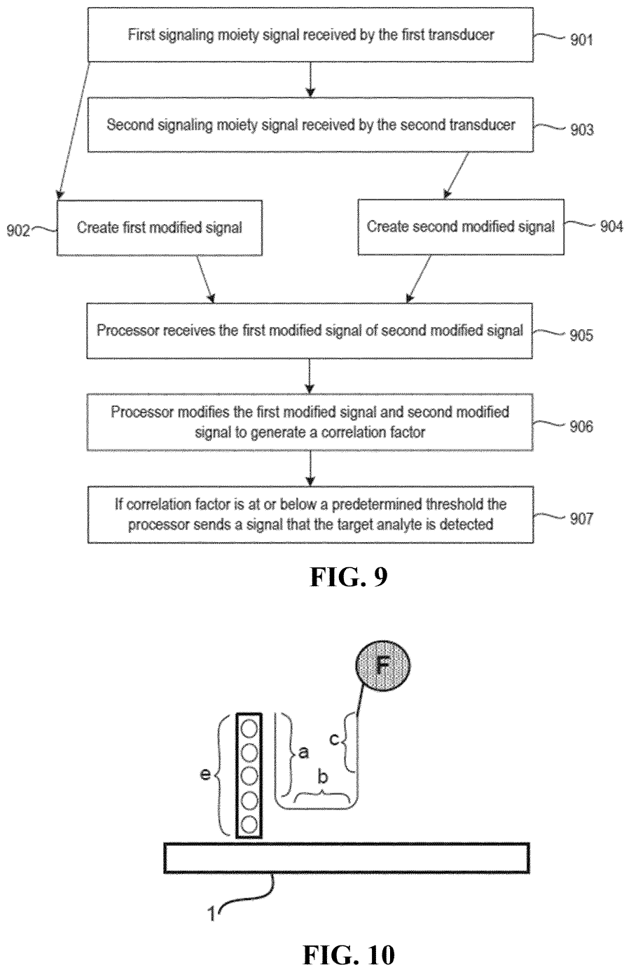

FIG. 9 is a flow chart illustrating how the first signaling moiety signal and second signaling moiety signal are correlated. The first transducer receives the first signal from the first signaling moiety 901. The first transducer may demodulate, descramble or decode the first signaling moiety signal to generate a first modified signal 902. The second transducer receives the second signal from the second signaling moiety 903. (Steps 901 and 903 or 902 and 903 may be simultaneous). The second transducer may demodulate, descramble or decode the second signaling moiety signal to generate a second modified signal 904. (Steps 901 and 904 or 902 and 904 may be simultaneous). In some embodiments, the steps 902 and 904 are omitted, i.e., the signal is not modified before being processed. The first and second modified signals may be sent to a processor (905). In some embodiments, the signals are not modified before being correlated. The processor may further modify the first and second modified signals (906). In some embodiments, the processor does not modify the first and second modified or unmodified signal but simply correlates the signals. The processor may modify the first and second modified signals by subtracting the first modified signal from the second modified signal (or vice versa) to determine a correlation factor. In some embodiments, when the correlation factor is at or above a predetermined standard, the processor sends a signal that the target analyte is detected (907). In some embodiments, only the first signal, or only the second signal, or both the first and second signal are modified prior to being received by the processor. In some embodiments, the first signal and second signal are not modified prior to being received by the processor. In some embodiments, only the first signal, or only the second signal, or both the first and second signal are modified by the processor to generate a correlation factor. In some embodiments, when the correlation factor is at or above a predetermined standard, the processor sends a signal that the target analyte is detected at a predetermined concentration. In some embodiments, when the correlation factor is at or above a predetermined standard, the processor sends a signal that the target analyte is detected at a predetermined concentration range. In some embodiments, when the correlation factor is below a predetermined standard, the processor sends a signal that the target analyte is not detected. In some embodiments, when the correlation factor is below a predetermined standard, the processor sends a signal that the target analyte is not detected at a predetermined concentration. In some embodiments, when the correlation factor is below a predetermined standard, the processor sends a signal that the target analyte is not detected at a predetermined concentration range. In some embodiments, when the correlation value is below a predetermine threshold a detection result is not reported, instead an error message is reported, i.e., the correlation value signals that the system did not process the sample properly.

In some embodiments, the first signaling moiety signal and second signaling moiety signal have a 1:1 relationship. In some embodiments, the first signaling moiety signal and second signaling moiety signal have a 1:1 or 1:2 or 1:3 or 1:4 or 1:5 or 1:10 or 1:20 or 1:50 or 1:100 relationship. In some embodiments, the first signaling moiety signal and second signaling moiety signal have a 2:1 or 2:3 or 2:4 or 2:5 or 2:10 or 2:50 relationship. In some embodiments, the first signaling moiety signal and second signaling moiety signal have a 3:1 or 3:2 or 3:4 or 3:5 or 3:10 or 3:20 or 3:50 or 3:100 relationship.

In some embodiments, the first signaling moiety signal and second signaling moiety signal are organized to form a new data stream. The new data stream is analyzed to determine a correlation factor. When the correlation factor is at or above a predetermined standard, the target analyte is detected. When the correlation factor is at or above a predetermined standard, the target analyte is detected at a predetermined concentration. In some embodiments, the correlation factor is based on one or more parameters selected from a group comprising/consisting of/consisting essentially of a correlation coefficient (R2), a scaled error of fit (EFT), a standard error of fit (RFT), slope of the line created by the new data stream, or intercept of the line created by the new data stream.

The processor may compare the first signaling moiety signal to a predetermined standard and compare the second signaling moiety signal to a second predetermined standard to determine a correlation factor. The processor may compare the first modified signal to a predetermined standard and compare the second modified signal to a second predetermined standard to determine a correlation factor.

In some embodiments, the result is based on the correlation value and the electrochemical signal value. In some embodiments, the result is based on the correlation value and the optical signal value. In some embodiments, the result is based on the correlation value, the electrochemical and optical signal value.

In some embodiments, the correlation is computed by multiplying two signals together and then summing the product. The result is a single number that indicates the similarity between the signals x[n] and y[n].

Herein, a correlation-based detection system is provided for correlating a first electrochemical signal and a second optical signal, the system comprising a correlation calculator configured to receive the first electrochemical signal and the second optical signal to produce a correlation value; a processor configured to evaluate the correlation value and determine whether a target analyte is present based on the correlation value and a predetermined value.

In some embodiments, both the electrochemical signal and the optical signal must be at or above a predetermined threshold in order for a detection result to be reported (detected or not detected). In some embodiments, the electrochemical signal but not the optical signal must be at or above a predetermined threshold in order for a detection result to be reported (detected or not detected). In some embodiments, the optical signal but not the electrochemical signal must be at or above a predetermined threshold in order for a detection result to be reported (detected or not detected). In some embodiments, the correlated signal (correlation value) must be at or above a predetermined threshold in order for a detection result to be reported (detected or not detected). In some embodiments, if correlated signal (correlation value) is at or below a predetermined threshold, an error signal is made. In some embodiments, if correlated signal (correlation value) is below a predetermined threshold, an error signal is made.
1. A method for generating a detection result the method comprising
   receiving by a first detector a first signal from a first signaling moiety;
   receiving by a second detector a second signal from a second signaling moiety;
   correlating the first signal and the second signal thereby generating a detection result.
2. The method of embodiment 1 wherein, correlating the first signal and the second signal comprises comparing the first signal to a predetermined standard to generate a first correlation signal and comparing the second signal to the first correlation signal.
3. The method of embodiment 1 wherein, correlating the first signal and the second signal comprises comparing the first signal to a first predetermined standard to generate a first correlation signal, comparing the second signal to a second predetermined standard to generate a second correlation signal and comparing the first correlation signal to the second correlation signal.
4. The method of embodiment 1 wherein, correlating the first signal and the second signal comprises adjusting the first signal based on an adjustment factor to generate a first correlation signal and comparing the second signal to the first correlation signal.
5. The method of embodiment 1 wherein, correlating the first signal and the second signal comprises adjusting the first signal based on a first adjustment factor to generate a first correlation signal, adjusting the second signal to a second adjustment factor to generate a second correlation signal and comparing the first correlation signal to the second correlation signal.
6. The method of embodiments 5 or 6 wherein the adjustment factor comprises removing the highest and lowest signals, the adjustment factor is the slope of the signal line, the adjustment factor is the amplitude of the signal line, or the adjustment factor is an estimate of the peak or valley of the signal.
7. The method of embodiments 1-6 wherein the first signaling moiety and second signaling moiety are from different modalities.
8. The method of embodiments 1-7 wherein the first signaling moiety detector and second signaling moiety detector are from different modalities.

### Methods

The signal probes disclosed herein can be used in methods of diagnosis, wherein the signal probe is complementary to a sequence (e.g., genomic or cDNA) of an infectious disease agent, e.g., of human disease including, but not limited to, viruses (e.g., HIV, HPV, etc.), bacteria, parasites, and fungi, thereby diagnosing the presence of the infectious agent in a sample from a patient. The type of target nucleic acid can be genomic, cDNA, mRNA, or synthetic, or the source may be human, animal, fungi or bacterial. In another embodiment that can be used in the diagnosis or prognosis of a disease or disorder, the target sequence is a wild-type human genomic DNA or RNA, or cDNA sequence, mutation of which is implicated in the presence of a human disease or disorder, or alternatively, can be the mutated sequence. In one embodiment, the same sample can be contacted with different sets of signal probes (for example, with differently labeled signal probes) which selectively identify the wild-type sequence or the mutated version. By way of example, the mutation can be an insertion, substitution, and/or deletion of one or more nucleotides, or a translocation. In another embodiment, the signal probes can be used in SNP analysis, pharmacogenomics and toxicogenetics.

In a specific embodiment, disclosed are methods for detecting or measuring a product of a nucleic acid amplification or synthesis reaction comprising: (a) contacting a sample comprising one or more target nucleic acid molecules with one or more signal probes (such probes comprising two or more labels, which are of a different modality and may be labeled internally, and/or, at or near the 3′- and/or at or near the 5′-end of the signal probe); and (b) detecting or measuring one or more targets by redox mediated electronic detection and optical detection. In some embodiments, the optical label is excited by an electrical voltage and the excitation is read by an optical reader. In some embodiments, the optical label is excited by an electrical voltage and the excitation is read by an electron transfer reader. In some embodiments, the ETM is excited by an electrical voltage and the excitation is read by an electron transfer reader. In some embodiments, the ETM label is excited by optical excitation (light or laser beam) and the excitation is read by an optical reader. In some embodiments, the ETM label is excited by optical excitation and the excitation is read by an electron transfer reader.

Disclosed are methods for detecting a target nucleic acid sequence, comprising contacting a sample containing a mixture of nucleic acids with at least one oligonucleotide, the oligonucleotide being capable of hybridizing a target nucleic acid sequence and comprising at least two detectable moieties (wherein the detectable moieties are of a different modality), wherein the first signaling moiety undergoes a redox reaction after an electrical voltage is applied to it, wherein the second signaling moiety undergoes an optical reaction after an electrical voltage is applied to it wherein a change in the redox potential indicates the presence of the target nucleic acid sequence and it wherein a change in the optical properties indicates the presence and/or concentration of the target nucleic acid sequence.

Disclosed are methods for detecting a target nucleic acid sequence, comprising contacting a sample containing a mixture of nucleic acids with at least one oligonucleotide, the oligonucleotide being capable of hybridizing a target nucleic acid sequence and comprising at least two detectable moieties (wherein the detectable moieties are of a different modality), wherein the first signaling moiety undergoes a redox reaction after an optical signal (light or laser beam) is applied to it, wherein the second signaling moiety undergoes an optical reaction after an optical signal (light or laser beam) is applied to it wherein a change in the redox potential indicates the presence of the target nucleic acid sequence and it wherein a change in the optical properties indicates the presence and/or concentration of the target nucleic acid sequence.

Disclosed are methods of determining the presence or absence of a target in a sample. In some embodiments, target sequence is a wild type human genomic or RNA or cDNA sequence. In some embodiments, target sequence is a mutated human genomic or RNA or cDNA sequence. The mutation is implicated in the presence of a human disease or disorder. In some embodiments, the signal probe amplifies the wild-type target and in others the mutated target. By way of example, the mutation can be an insertion, substitution, and/or deletion of one or more nucleotides, or a translocation. In another embodiment, the signal probes can be used in SNP analysis, pharmacogenomics and toxicogenetics.

In a specific embodiment, disclosed is a method for detecting the presence or absence of a target nucleic acid comprising: (a) contacting a sample comprising one or more target nucleic acid molecules with one or more signal probes (such probes may comprise two or multiple labels, where at least two are of a different modality but others may be the same and may be labeled internally, and/or, at or near the 3′- and/or at or near the 5′-end of the signal probe); and (b) detecting or measuring one or more target nucleic acid molecules by electrochemical detection and detecting or measuring one or more target nucleic acid molecules by optical detection.

In a specific embodiment, disclosed is a method for detecting the presence or absence of a target nucleic acid comprising: (a) contacting a sample comprising one or more target nucleic acid molecules with one or more signal probes (such probes may comprise two or multiple labels, where at least two are of a different modality but others may be the same and may be labeled internally, and/or, at or near the 3′- and/or at or near the 5′-end of the signal probe); and (b) detecting or measuring one or more target nucleic acid molecules by a single detection method. Wherein the single detection method is electrochemical detection or optical detection.

Disclosed herein are methods for determining the absence of at least one particular target or template nucleic acid molecule in a sample, comprising: (a) contacting a sample comprising one or more target nucleic acid molecules with one or more signal probes (such probes may comprise two or multiple labels, where at least two are of a different modality but others may be the same and may be labeled internally, and/or, at or near the 3′- and/or at or near the 5′-end of the signal probe); and (b) failing to detect or measure one or more target nucleic acid molecules by electrochemical detection and failing to detect or measure one or more target nucleic acid molecules by optical detection. Such failure to detect indicates the absence of the at least one particular target or template nucleic acid molecule in the sample.

Disclosed herein are methods for determining the absence of at least one particular target or template nucleic acid molecule in a sample, comprising: (a) contacting a sample comprising one or more target nucleic acid molecules with one or more signal probes (such probes may comprise two or multiple labels, where at least two are of a different modality but others may be the same and may be labeled internally, and/or, at or near the 3′- and/or at or near the 5′-end of the signal probe); and (b) failing to detect or measure one or more target nucleic acid molecules by a single detection method. Wherein the single detection method is electrochemical detection or optical detection. Such failure to detect indicates the absence of the at least one particular target or template nucleic acid molecule in the sample.

In a further aspect, the invention provides methods of detecting a target analyte in a sample. The methods comprise adding the sample to a composition as outlined above, such that the target analyte binds to the transport composition (signaling probe) and the reporter composition (capture probe) to form an assay complex and the presence or absence of the first signaling moiety (e.g., an ETM, such as ferrocene, a ferrocene derivative,, methylene blue, osmium signaling moiety) and second signaling moiety (e.g., optical signaling moiety such as FM) are detected wherein the first signaling moiety and second signaling moiety are different modalities. In some embodiments, the detector is an ETM detector, an optical detector or both. In some embodiments, the ETM and OSM are excited by an electrical voltage. In some embodiments, the ETM and OSM are excited by a light or laser beam source. In some embodiments, the ETM is excited by a light or laser beam source and the OSM is excited by an electrical voltage. In some embodiments, the OSM is excited by a light or laser beam source and the ETM is excited by an electrical voltage.

Stated in more detail, target nucleic acids are extracted from samples and amplified using Polymerase Chain Reaction (PCR). The resulting double-stranded DNA is then digested by an exonuclease to create a single-stranded DNA. The single-stranded DNA is bound to a signal probe that has a first signaling moiety (e.g., an ETM, such as ferrocene, a ferrocene derivative, methylene blue, or osmium) and second signaling moiety (e.g., an optical signaling moiety, such as FM) wherein the first signaling moiety and second signaling moiety are different modalities (“signal complex”). Binding the signal complex with a capture probe (“hybridized complex”) brings the first signaling moiety (e.g., ETM such as ferrocene) and second signaling moiety (e.g., optical signaling moiety such as FM) near the surface of the electrode (gold, silver, platinum electrode). In some embodiments, an electric voltage, specific to the first signaling moiety (e.g., ETM such as ferrocene) is applied. In some embodiments, an electric voltage, specific to the second signaling moiety (e.g., optical signaling moiety such as FM) is applied (i.e., a single voltage excites one or the other of the ETM or optical signaling moieties but not both). In some embodiments, an electric voltage, specific to the hybridized complex is applied (i.e., a single voltage excites both the ETM and optical signaling moieties). An electric signal, specific to the first signaling moiety (e.g., ETM such as ferrocene), is generated as a byproduct of a reduction-oxidation reaction when voltage is applied to the system (FIG. 1a). A first detection instrument measures and interprets this electrical output (e.g., in nanoamps, nA, uA, mA etc.) to determine the results for each target (Detected or Not Detected). An optical/fluorescent signal, specific to the second signaling moiety (e.g., optical signaling moiety such as a FM), is generated as a byproduct when voltage is applied to the system. A second detection instrument measures and interprets this fluorescent output (in wavelengths) to determine the results for each target (Detected, Not Detected, or the concentration of the target). In some embodiments, the first detection instrument measures and interprets the fluorescent output from the OSM (e.g., in nanoamps, nA, uA, mA etc.) to determine the results for each target (Detected, Not Detected, or the concentration of the target).

In some embodiments, a light or laser beam, specific to the first signaling moiety (e.g., ETM such as ferrocene) is applied. In some embodiments, a light or laser beam, specific to the second signaling moiety (e.g., optical signaling moiety such as a FM) is applied (i.e., a single wavelength excites one or the other of the ETM or optical signaling moieties but not both). In some embodiments, a light or laser beam specific to the hybridized complex is applied (i.e., a single wavelength excites both the ETM and optical signaling moieties). An electric signal, specific to the first signaling moiety (e.g., ETM such as ferrocene), is generated as a byproduct of a reduction-oxidation reaction when a light or laser beam is applied to the system. A first detection instrument measures and interprets this electrical output (e.g., in nanoamps, nA, uA, mA etc.) to determine the results for each target (Detected or Not Detected). An optical/fluorescent signal, specific to the second signaling moiety (e.g., optical signaling moiety such as a FM), is generated as a byproduct when light or laser beam is applied to the system. A second detection instrument measures and interprets this fluorescent output (in wavelengths) to determine the results for each target (Detected, Not Detected, or the concentration of the target). In some embodiments, the second detection instrument measures and interprets the signal from the ETM (in wavelengths) to determine the results for each target (Detected or Not Detected).

Disclosed is the use of “signal probes” that have a ferrocene tag (N6, QW56 or QW80) toward, near, or at the 5′-end and a second label located toward, near, or at the 5′-end. Disclosed is the use of “signal probes” that have a ferrocene tag (N6, QW56 or QW80) toward, near, or at the 5′-end and a second label located elsewhere. In some embodiments, the second label is an optical label such as a colorimetric, luminescent, or fluorescent label such as ethidium bromide, fluorescein, or green fluorescent protein. As shown in FIG. 1b, the signal probe will bind to a capture probe and bring the ferrocene label(s) and optical label(s) near the surface of the electrode to generate a first electrical signal and a second fluorescent signal when a voltage is applied. In some embodiments, the first electrical signal is detected by an electrochemical detector and a second fluorescent signal is detected by a fluorescent detector. In some embodiments, the first electrical signal is detected by an electrochemical detector and a second fluorescent signal is detected by the electrochemical detector. In some embodiments, the first electrical signal is detected by a fluorescent detector and a second fluorescent signal is detected by the fluorescent detector.

In some embodiments, the signal probe will bind to a capture probe and bring the ferrocene label(s) and optical label(s) near the surface of the electrode to generate a first electrical signal and a second fluorescent signal when a light or laser beam is applied. In some embodiments, the first electrical signal is detected by an electrochemical detector and a second fluorescent signal is detected by a fluorescent detector. In some embodiments, the first electrical signal is detected by a fluorescent detector and a second fluorescent signal is detected by the electrochemical detector. In some embodiments, the first electrical signal is detected by a fluorescent detector and a second fluorescent signal is detected by the fluorescent detector.

The methods disclosed herein may be better understood by the following numbered paragraphs.
Paragraph 1: A method of performing a nucleic acid detection comprising:
   (a) combining reagents for a polymerase chain reaction, DNA polymerase, a target nucleic acid, and a primer;
   (b) cycling the mixture of (a) to provide multiple copies of an amplicon;
   (c) exposing the mixture in (b) to an exonuclease to produce a single stranded amplicon;
   (d) exposing said mixture in (c) to a signal probe oligonucleotide complementary to said single stranded amplicon, wherein the signal probe oligonucleotide comprises a first electrochemical detectable label and an optically detectable label and thereby hybridizing said signal probe oligonucleotide to the single stranded amplicon;
   (e) exposing said mixture in (d) to a capture probe oligonucleotide complementary to said single stranded amplicon wherein the capture probe oligonucleotide is bound to an electrode surface and thereby hybridizing said mixture in (d) to the capture probe oligonucleotide;
   (f) applying a voltage to the electrode surface; and
   (g) detecting said electrochemically detectable label and the optically detectable label.
Paragraph 2. The method of Paragraph 1, wherein said target nucleic acid is selected from the group consisting of deoxyribonucleic acid and ribonucleic acid and modifications and derivatives thereof.
Paragraph 3. The method of Paragraph 1, wherein said target nucleic acid is extracted from blood, a buccal swab, tissue, a bodily fluid, an environmental sample, a surface of a material, a plant, an animal, a bacteria, or a fungi.
Paragraph 4. The method of Paragraph 1, wherein said electrochemically detectable label is an electron transfer moiety.
Paragraph 5. The method of Paragraph 1, wherein said electrochemically detectable label is a ferrocene label.
Paragraph 6. The method of Paragraph 1, wherein cycling is isothermal.
Paragraph 7. The method of Paragraph 1, wherein said capture probe oligonucleotide is immobilized on a gold surface or carbon-based surface.
Paragraph 8. The method of Paragraph 1, wherein said detection is electrochemical.
Paragraph 9. The method of Paragraph 1, wherein said detection is optical.
Paragraph 10. The method of Paragraph 1, wherein said optical label is not exposed to a second moiety, which has a second label or a quencher.

The methods disclosed herein may be better understood by the following numbered paragraphs.
Paragraph 1. A method of performing a nucleic acid detection comprising:
   (a) exposing a sample to a first signal probe having a first oligonucleotide sequence, a first detectable label and a second detectable label wherein the first detectable label and the second detectable label are of a different modality;
   (b) exposing the sample to a second signal probe having a first and a second oligonucleotide sequence, a first detectable label and a third detectable label wherein the first detectable label and the second detectable label are of a different modality wherein the second and third detectable label are of a different modality and wherein the first oligonucleotide sequence and second oligonucleotide sequence are of a different modality;
   (c) exposing the mixture in (a and b) to a first capture oligonucleotide complementary to said first signal probe and a second capture oligonucleotide complementary to said second signal probe;
   (d) hybridizing said signal probes, with said capture oligonucleotides; and
   (e) detecting said label associated with said (i) first detectable label and not the second or third detectable label, (ii) first and second detectable labels and not the third detectable label (iii) first and third detectable labels and not the second detectable label, or (iv) second and third detectable label and not the first detectable label.

### Method for Detecting a Targeted Pathogen

Disclosed is a method for detecting a targeted pathogen in a sample with the use of a photodetector and electrochemical detector, the method comprising:
a. providing a system comprising:
   i. immobilized capture DNA sequence probe positioned on a surface substrate, wherein the substrate is an electrode or is connected to an electrode, wherein the immobilized capture DNA sequence probe having a DNA sequence complementary to a known DNA sequence of the target pathogen; and
   ii. capture DNA sequence probe having a DNA sequence complementary to a known DNA sequence of the target pathogen, wherein the free capture DNA sequence probe has attached thereto a fluorophore or equivalent thereof and an electron transfer moiety (ETM);
b. contacting the immobilized capture DNA sequence probe with sample comprising target pathogen, wherein any DNA sequence of the target pathogen binds to the immobilized capture DNA sequence probe;
c. contacting the bound DNA sequence of the target pathogen with the free capture DNA sequence probe, wherein binding of the free capture DNA sequence probe to the DNA sequence of the target pathogen causes the fluorophore or equivalent thereof to be positioned from about 5 nm to about 50 nm from the surface substrate;
d. applying an electrical voltage to the electrode thereby exciting the fluorophore or equivalent thereof; and
e. measuring the fluorescence of the fluorophore or equivalent thereof with a photodetector and measuring the current of the ETM with a current flow detector, wherein the current is not proportional to the amount of ETM and, wherein the fluorescence is proportional to the amount of fluorophore or equivalent thereof.

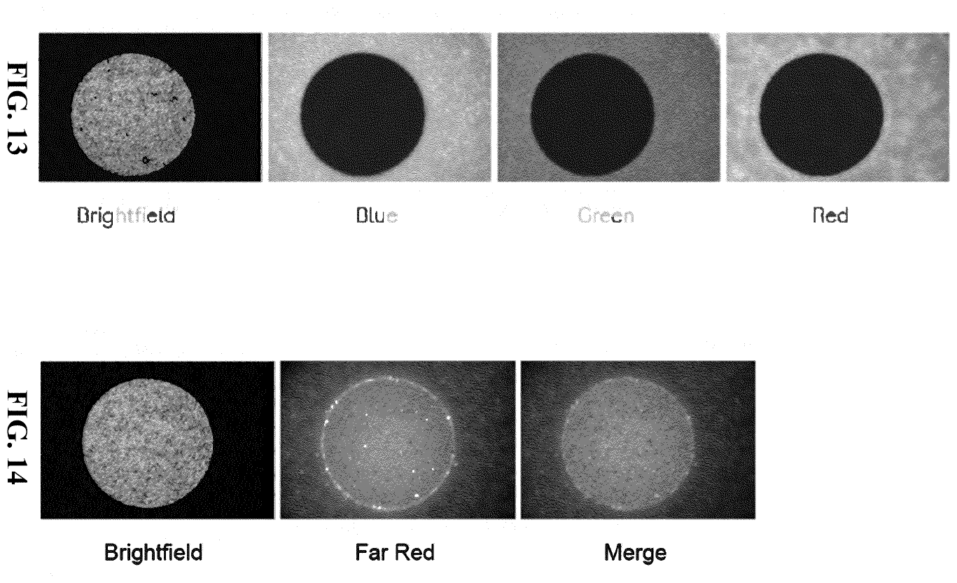

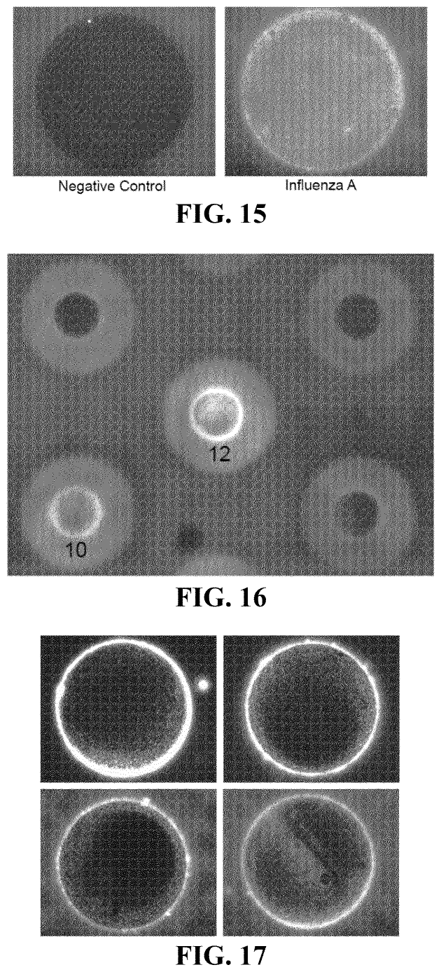

The data demonstrates that there is no significant interference/quenching caused by the gold electrode. See FIG. 15 and FIG. 16.

The substrate may include any form of metals such as silver, gold, platinum, zinc, aluminum, indium, palladium, rhodium iron, nickel, copper, carbon, and combination thereof and more preferably the substrate is gold. The substrate can include, glass, quartz, cellulose and/or a polymeric material.

In some embodiments, at least a portion of each capture probe-signal probe-target hybridization complex is in contact with a polar solvent or a dipolar aprotic solvent that has a dipole moment and inducible, such as water, other polar solvents, including methanol or acetic acid, ionic salt solutions and/or acetone, ethylene acetate.

The system thus requires post-amplification processing before detecting the OSM.

### Assay

Disclosed is an assay, the method comprising:
a. providing at least one vessel or container; wherein a first and second electrode are positioned within the vessel or communicatively connected thereto;
b. introducing capture probes into the vessel, wherein the vessel includes a polar solution, and wherein the capture probes are connected to a surface of the vessel and communicatively connected to the first and second electrodes;
c. introducing a molecule that exhibits signaling upon excitation with electrical voltage and disposing such molecule near the capture probe, wherein the molecule is positioned at a predetermined proximity to an electrode surface to; and
d. measuring signal upon excitation with electrical voltage with an optical detector.

### Real-Time Detection

Real-time PCR (also known as quantitative PCR (qPCR)), in contrast to the regular PCR, offers the ability to detect amplification reaction in real time. The reaction kinetics can be monitored in the liquid phase while the amplification process is still proceeding. Real-time chemistries allow for the detection of PCR amplification during the early phases of the reaction. Based on the increase of the fluorescence intensity from a specific dye, the concentration of the target can be determined even before the amplification reaches its plateau.

Disclosed is a method for real-time measuring of the amounts of the amplicon in PCR, using a signal probe with one or more electro-active indicators (oxidation moieties, reduction moieties, redox moieties and/or transition metal complex) and one or more optically active indicators (such as ethidium bromide, fluorescein, or green fluorescent protein).

Accordingly, one aspect of the present subject matter is directed to a first signaling moiety useful for electrochemically detecting the amplified nucleic acid and a second signaling moiety for quantifying the amplified nucleic acid in real-time or after each PCR thermal cycle, comprising: contacting an amplified sample comprising a target nucleic acid with a dual-labeled signal probe (labeled with at least one electroactive indicator, and at least one optical label) adding an electric potential, and detecting or measuring in real-time or after each PCR thermal cycle an electric signal produced by the electroactive indicator and the optical indicator thereby quantifying the amount of nucleic acid present in the sample. In some embodiments, no optical signal is applied to the complex to excite the OSM.

In one aspect, is a real time solid phase method for electrochemically or electrically monitoring or quantifying the amount of nucleic acid(s) by formation of a polymerase chain reaction (PCR) produced polynucleic acid(s) that comprises the following steps:
a. contacting a sample comprising a target nucleic acid(s), with polymerase chain reaction enzyme(s) under conditions effective for PCR amplification to occur;
b. amplifying target nucleic acid(s);
c. dissociating the amplified target nucleic acid(s) to form single stranded target nucleic acid(s);
d. hybridizing the single stranded target nucleic acid(s) with signal probes, the signal probes comprising a first ETM and a second optical label to form a signal-target hybridization complex;
e. hybridizing the signal-target hybridization complex with capture probes bound to an electrode;
f. applying an electric potential to the sample and detecting or measuring in real time the signal(s) produced by the optical label; and
g. quantifying the amount of nucleic acid(s) present in the sample and the amount of polynucleic acid(s) produced by correlating the change(s) in optical signal(s) over time with the formation of polynucleic acid(s).

The electrode may comprise a surface comprising a solid support; the solid support may comprise glass, and the surface of at least one electrode(s), being patterned and integrated into the microchip, comprise(s) indium tin oxide, gold, or platinum. The microchip may further comprise a temperature sensor(s) and a micro heater(s) integrated therein. The temperature sensor(s) and the micro heater(s) may also be off-chip. In some embodiments, the device is a portable device and/or microdevice.

By coupling an electrochemical detection method with an optical detection method, a superior method of detection and quantification of target molecules, such as nucleic acids or nucleic acid coupled molecules, which is less costly, simpler, and more accurate than prior art methods is disclosed.

The methods described herein can be more fully understood by reference to the following numbered paragraphs:
Paragraph 1. An amplification method for the real-time analysis of nucleic acid samples comprising the steps of contacting said sample with a nucleic acid primer or probe capable of hybridizing with a selected target nucleic acid under chain extension conditions; conducting polymerase mediated chain extension to produce a PCR product wherein the PCR product does not have a detectable label; conducting post-PCR processing wherein a detectable label is hybridized to the PCR product; detecting the presence or absence of a detectable signal based on the presence or absence of the target nucleic acid in a sample.
Paragraph 2. The method of paragraph 1 wherein the post-PCR processing comprises contacting the PCR product with a signal probe labeled with a FM to produce a signal probe/PCR product complex.
Paragraph 3. The method of paragraph 2, wherein the post-PCR processing further comprises contacting the signal probe/PCR product complex with a capture probe to produce a signal probe/ PCR product/capture probe complex.
Paragraph 4. The method of paragraph 3, wherein the post-PCR processing further comprises contacting the signal probe/PCR product complex with a capture probe to produce a signal probe/ PCR product/capture probe complex.
Paragraph 5. The method of paragraph 4, wherein the post-PCR processing further comprises applying an electrical voltage to the signal probe/PCR product/capture probe complex thereby detecting the presence or absence of the target nucleic acid in the sample.

The methods described herein can be more fully understood by reference to the following numbered paragraphs:
Paragraph 1. A method for the detection of a target nucleic acid in a sample, comprising providing a reaction mixture comprising a target nucleic acid, at least one oligonucleotide primer, a DNA polymerase; amplifying the target nucleic acid; providing at least one oligonucleotide probe wherein the probe comprises an optical signaling moiety and hybridizing the at least one oligonucleotide probe to the amplified target nucleic acid; and detecting the complex, wherein presence of the complex is indicative of the presence of the target nucleic acid in the sample.
Paragraph 2. The method of embodiment 1, wherein the target nucleic acid comprises at least one of DNA and RNA.
Paragraph 3. The method of any prior paragraph, wherein the target nucleic acid is RNA and amplifying of the target nucleic acid includes a stage wherein at least one DNA copy of said RNA is synthesized using a reverse transcriptase.
Paragraph 4. The method of any prior paragraph, wherein a plurality of target nucleic acids is amplified and detected.
Paragraph 5. The method of embodiment 4, wherein at least one oligonucleotide primer and at least one oligonucleotide probe is provided for every target nucleic acid amplified and detected.
Paragraph 6. The method of embodiment 4, wherein more than one of the oligonucleotide primers is provided for every said target nucleic acid amplified and detected.
Paragraph 7. The method of any prior paragraph, wherein detection of the target nucleic acid is performed after the amplification.
Paragraph 8. The method of any prior paragraph, wherein detection of the target nucleic acid is performed in real time.
Paragraph 9. The method of any prior paragraph, wherein detection comprises hybridizing the complex to a capture probe wherein the capture probe is bound to an electrode.
Paragraph 10. The method of any prior paragraph, wherein detection comprises hybridizing the complex to a capture probe wherein the capture probe is immobilized on an electrode and applying an electrical voltage to the electrode.
Paragraph 11. The method of any prior paragraph, wherein detection comprises hybridizing the complex to a capture probe wherein the capture probe is immobilized on an electrode and applying an electrical voltage to the electrode and detecting the signal via an optical detector.
Paragraph 12. The method of any prior paragraph, wherein the oligonucleotide probe further comprises an ETM, and detection comprises hybridizing the complex to a capture probe wherein the capture probe is bound to an electrode and applying an electrical voltage to the electrode and detecting the signal via an optical detector and ETM detector.
Paragraph 13. The method of any prior paragraph, wherein amplifying and detecting of the target nucleic acid is performed to measure the amount of said target nucleic acid in said sample.
Paragraph 14. The method of any prior paragraph, wherein amplifying of the target nucleic acid comprises use of isothermal amplification.
Paragraph 15. The method of any prior paragraph, wherein said optical signaling moiety is a fluorescent label.
Paragraph 16. The method of any prior paragraph, wherein detection comprises detecting a detection signal, and wherein detection of the signal is indicative of the presence of the target nucleic acid in the reaction mixture.
Paragraph 17. The method of any prior paragraph, wherein a plurality of target nucleic acids is amplified and detected, and wherein at least one said oligonucleotide primer is provided for every target nucleic acid amplified and at least one said oligonucleotide probe is provided for every target nucleic acid detected.
Paragraph 18. The method of any prior paragraph, wherein the optical label comprises at least one of a fluorescent label, a fluorescence-polarization label, or FRET probe, wherein the FRET probe changes its fluorescent properties upon forming the complex with the target nucleic acid, and wherein the changes are indicative of the presence of the complex.
Paragraph 19. The method of embodiment 18, wherein the FRET probe comprises a hybridization-triggered FRET probe.
Paragraph 20. The method of embodiment 19, wherein the hybridization-triggered FRET probe comprises at least one of a Scorpion primer and a Beacon probe.
Paragraph 21. The method of embodiment 20, wherein the FRET probe comprises at least one of a cleavable FRET probe and a cleavable FRET probe comprising a TaqMan probe.
Paragraph 22. The method of any prior paragraph, wherein the detection PCR comprises quantitative PCR.

### Determining Target Concentration

As discussed above, electrochemical detection is an end-point detection system. It cannot be used to determine the concentration of a target mid-amplification. Adding a second optical label to the signal probe, which can be detected mid-amplification cycle converts an end-point detection system into a real-time detection system.

Disclosed is a detection system for detecting the concentration of a target analyte comprising a cartridge, the cartridge comprising a first measurement unit comprising an optical measuring instrument configured to measure concentration of the target analyte, wherein the first measurement unit measures a concentration of the target analyte using an optical method subsequent to exciting an optical signaling moiety with a voltage; a second measurement unit comprising an electrochemical measuring instrument configured to measure the existence but not the concentration of the target analyte, wherein the second measurement unit measures the existence of the target analyte using an electrochemical method subsequent to exciting an ETM with a voltage.

In some embodiments, the optical measuring instrument does not have an optical excitation mechanism (such as a light source) to excite the optical signaling moiety.

In some embodiments, the optical signaling moiety/fluorescent moiety is excited by the same electrical voltage that excites the ETM. In some embodiments, the optical signaling moiety/fluorescent moiety is excited by a different electrical voltage than excites the ETM.

Disclosed is a method for detecting the concentration of a target analyte comprising binding target analyte to a signal probe; obtaining a first measurement from an optical signaling moiety, wherein the first measurement measures a concentration of the target analyte using an optical method; obtaining a second measurement from an ETM, wherein the second measurement does not measure a concentration of the target analyte using an optical method but measure the presence of the target analyte using an electrochemical method.

Disclosed are methods for quantifying a target nucleic acid molecule, comprising contacting a sample containing a mixture of nucleic acids comprising the target nucleic acid molecule with at least one oligonucleotide containing a first signaling moiety and a second signaling moiety, wherein the first signaling moiety undergoes a detectable redox reaction upon application of an electrical voltage, wherein the second signaling moiety undergoes a detectable optical reaction upon application of an electrical voltage and observing the reaction, wherein the observable reaction is proportional to the amount of the target nucleic acid molecule in the sample.

### Multiplex compositions

A multiplex assay is a type of assay that simultaneously measure multiple analytes in a single experiment. Adding an optical signaling moiety to the signal probe increases the detection systems multiplex capabilities.

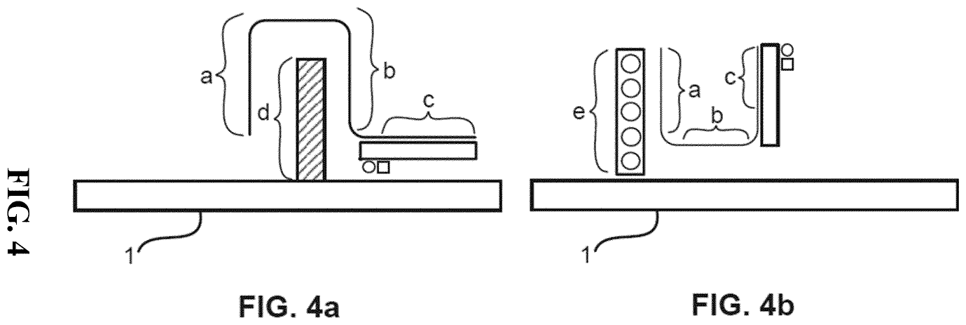

Aspects of this disclosure relate to a method of determining which of a plurality of different target nucleic acids are present in a test sample. In some embodiments, different targets are detected on different electrodes because different capture probes with different binding affinities are attached to different electrodes (See FIGS. 4a and 4b). Additionally, multiplexing can be enhanced by using the optical signaling moiety for a control and the ETM for detection. In some embodiments, the ETM is used for a control and the optical signaling moiety for detection.

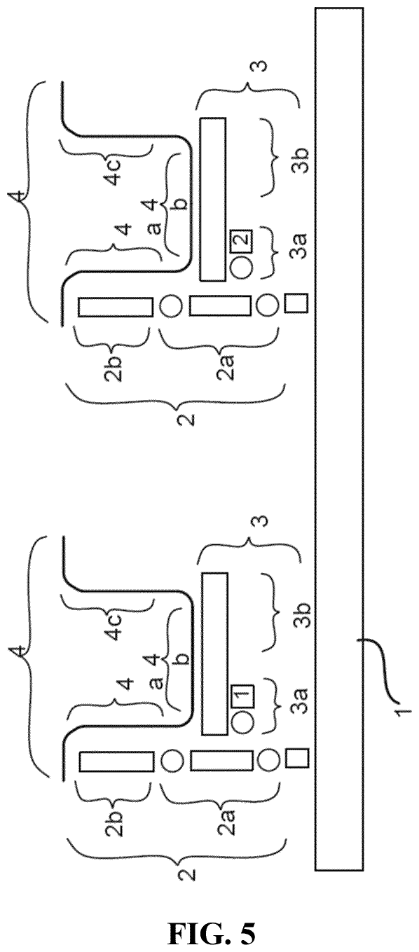

In some embodiments, the methods include contacting the sample with multiple different signal probes wherein the first signal probe comprises an ETM and a first optical signaling moiety and the second signal probe comprises an ETM and a second optical signaling moiety wherein the first optical signaling moiety and second optical signaling moiety are of a different modality and can be distinguished from one another. See FIG. 5.
Paragraph 1. A device for the multiplexed detection of a target analyte comprising:
   a) a first signal probe comprising an ETM and a first optical signaling moiety;
   b) a second signal probe comprising an ETM and a second optical signaling moiety;
   c) a capture probe;
   d) a first detector capable of detecting a signal produced by the ETM;
   e) a second detector capable of distinguishing a first optical signal produced by the first optical signaling moiety from the second optical signal produced by the second optical signaling moiety thereby detecting a target.
Paragraph 2. A method for the multiplexed detection of a target analyte in a sample comprising:
   a) exposing a first signal probe comprising an ETM and a first optical signaling moiety to the sample;
   b) exposing a second signal probe comprising an ETM and a second optical signaling moiety to the sample;
   c) hybridizing the first signal probe to a first portion of the sample to produce a first signal-target hybridization complex;
   d) hybridizing the second signal probe to a second portion of the sample to produce a second signal-target hybridization complex;
   e) hybridizing the first signal-target hybridization complex to a first capture probe to form a first capture probe hybridization complex;
   f) hybridizing the second signal-target hybridization complex to a second capture probe to form a second capture probe hybridization complex;
   g) applying a voltage to the first capture probe hybridization complex and the second capture probe hybridization complex;
   d) detecting the optical signal from the first capture probe hybridization complex and the second capture probe hybridization complex;
   e) detecting the electrochemical signal from the first capture probe hybridization complex and the second capture probe hybridization complex thereby detecting a target analyte in a sample.

### SNP Analysis by Fluorescencetection

Methods, kits, and signal probes for detecting a single nucleotide polymorphism (SNP) in a target nucleic acid in a sample are described herein. The increased sensitivity of real-time PCR for detection of a SNP in a target nucleic acid compared to other methods, as well as the improved features of real-time PCR including sample containment and real-time detection of the amplified product, make feasible the implementation of this technology for routine diagnosis and detection of a SNP in a target nucleic acid in the clinical laboratory. Methods provided avoid problems of sample contamination, false negatives, and false positives.

The methods may include performing at least one cycling step that includes amplifying one or more portions of a target nucleic acid molecule, e.g., a gene target containing the SNP of interest to be detected, in a sample using one or more primers or one or more primer pairs. The primers specifically anneal to the nucleic acid sequence target, and initiate synthesis therefrom under appropriate conditions. Each of the primers anneal to a region within or adjacent to the respective target nucleic acid molecule such that at least a portion of each amplification product contains nucleic acid sequence corresponding to respective target and SNP, if present. An amplification product is produced provided that the target nucleic acid is present in the sample, whether or not the SNP of interest is present in the target nucleic acid molecule.

The method can also include a hybridizing step that includes contacting the amplification product with a SNP specific signal probe including a nucleic acid sequence complementary to a SNP containing region of the amplification product. The SNP specific signal probe can include a first detectable label and a second detectable label (wherein the first detectable label and the second detectable label are of different modalities). The signal probe can be designed to include a nucleic acid region that is non-naturally occurring which may include one or more changed nucleotides that are not part of the naturally occurring sequence, or may include one or more additional non-naturally occurring nucleotides, which are nucleotides added to the naturally occurring sequence. In order to detect whether or not the SNP of interest is present or absent in the nucleic acid target in the sample, the amplification product is detected by way of the detectable label being activated on the signal probe. If the amplification product is detected by way of the SNP specific signal probe, the presence of SNP is indicated. If alternatively, the amplification product is not detected by way of the SNP specific signal probe, the presence of SNP is not indicated. Thus, the presence of the amplification products is indicative of the presence of the SNP in the target nucleic acid target, and the absence of the amplification products is indicative of the absence of the SNP in the target nucleic acid target.

For detection of SNP in the target nucleic acid sequence, primers, and probes to amplify the target nucleic acid sequences can be prepared. Also, functional variants can be evaluated for specificity and/or sensitivity by those of skill in the art using routine methods. Representative functional variants can include, e.g., one or more deletions, insertions, and/or substitutions in the primers and/or probes disclosed herein. For example, a substantially identical variant of the primers or probes can be provided in which the variant has at least, e.g., 80%, 90%, or 95% sequence identity to one original primers and probes, or a complement thereof.

A functionally active variant of any of primer and/or probe may be identified which provides a similar or higher specificity and sensitivity in the presently described methods, kits, or signal probes as compared to the respective original sequences.

As described herein, amplification products can be detected using labeled signal probes that take advantage of FRET technology. One FRET format utilizes TaqMan® technology to detect the presence or absence of an amplification product, and hence, the presence or absence of a SNP in a target nucleic acid. TaqMan® technology utilizes one single-stranded hybridization signal probe labeled with two fluorescent moieties. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target DNA (i.e., the amplification product) and is degraded by the 5′ to 3′ exonuclease activity of the Taq polymerase during the subsequent elongation phase. As a result, the excited fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology and is suitable for performing the methods described herein for detecting the presence or absence of a SNP in a target nucleic acid in the sample. Roche's Lightcycler or COBAS can also detect a SNP in a target.

Generally, the presence of FRET indicates the presence of the SNP in a target nucleic acid in the sample, and the absence of FRET indicates the absence of HSV-1 and/or HSV-2 in the sample. Inadequate specimen collection, transportation delays, inappropriate transportation conditions, or use of certain collection swabs (calcium alginate or aluminum shaft) are all conditions that can affect the success and/or accuracy of a test result, however. Using the methods disclosed herein, detection of FRET within, e.g., 45 cycling steps is indicative of the presence of an SNP in a target nucleic acid in a sample.

Representative biological samples that can be used in practicing the methods of the invention include, but are not limited to dermal swabs, nasal swabs, wound swabs, blood cultures, skin, and soft tissue infections. Collection and storage methods of biological samples are known to those of skill in the art. Biological samples can be processed (e.g., by nucleic acid extraction methods and/or kits known in the art) to release target nucleic acid or in some cases, the biological sample can be contacted directly with the PCR reaction components and the appropriate oligonucleotides. Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples. Each thermocycler run can also include a negative control that, for example, lacks target template DNA. Such controls are indicators of the success or failure of the amplification, hybridization, and/or FRET reaction. Therefore, control reactions can readily determine, for example, the ability of primers to anneal with sequence-specificity and to initiate elongation, as well as the ability of probes to hybridize with sequence-specificity and for FRET to occur.

In an embodiment, the methods of the invention include steps to avoid contamination. For example, an enzymatic method utilizing uracil-DNA glycosylase is described in U.S. Pat. Nos. 5,035,996; 5,683,896; and 5,945,313 to reduce or eliminate contamination between one thermocycler run and the next.

Conventional PCR methods in conjunction with FRET technology can be used to practice the methods of the invention. In one embodiment, a LightCycler® instrument is used. The following patent applications describe real-time PCR as used in the LightCycler® technology: WO 97/46707, WO 97/46714, and WO 97/46712.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. After each cycling step, a quantitative display of fluorescence vs. cycle number can be continually updated for all samples. The data generated can be stored for further analysis.

It is understood that the embodiments of the present invention are not limited by the configuration of one or more commercially available instruments.
Paragraph 1. A method for detecting a single nucleotide polymorphism (SNP) in a target nucleic acid in a sample, the method comprising:
   performing an amplifying step comprising contacting the sample with a primer comprising a first nucleic acid sequence to produce an amplification product comprising a region containing the SNP if the target nucleic acid is present in the sample;
   adding a SNP specific signal probe comprising a second nucleic acid sequence complementary to the region containing the SNP of the amplification product into the sample and performing a hybridizing step comprising contacting the amplification product with the SNP specific signal probe, if the target nucleic acid is present in the sample, the SNP specific signal probe comprising an ETM and a fluorescent moiety; and
   detecting the presence or absence of the amplification product, wherein the presence of the amplification products is indicative of the presence of the SNP in the target nucleic acid.
Paragraph 2. A kit for detecting a single nucleotide polymorphism (SNP) in a target nucleic acid in a sample, comprising:
   at least one primer comprising a first nucleic acid sequence specific to produce an amplification product comprising a region containing the SNP if the target nucleic acid is present in the sample; and
   a SNP specific signal probe comprising a second nucleic acid sequence complementary to the region containing the SNP of the amplification product, the SNP specific signal probe comprising an ETM and a fluorescent moiety.
Paragraph 3. A single nucleotide polymorphism (SNP) specific signal probe comprising a nucleic acid sequence complementary to a SNP containing region of an amplification product, the SNP specific signal probe comprising an ETM and a fluorescent moiety.
Paragraph 4. The method, kit, or signal probe of Paragraphs 1-3, wherein the donor fluorescent moiety comprises a donor fluorescent moiety and an acceptor moiety of the donor fluorescent moiety.
Paragraph 5. The method, kit, or signal probe of Paragraph 4, wherein the acceptor moiety is in an internal position of the SNP specific hydrolysis probe.
Paragraph 6. The method, kit, or signal probe of Paragraph 4, wherein the ETM is at the 5′ end, donor fluorescent moiety is at the 5′ end, and the acceptor moiety is within no more than 5 nucleotides from the donor fluorescent moiety on the signal probe.
Paragraph 7. The method, kit, or signal probe of Paragraph 4, wherein the acceptor moiety is a quencher.
Paragraph 8. The method of Paragraph 1, wherein the amplifying step employs a polymerase enzyme having 5′ to 3′ exonuclease activity.
Paragraph 9. The method, kit, or signal probe of any proceeding Paragraphs, wherein the first nucleic acid sequence of the primer and/or the second nucleic acid sequence of the signal probe comprises/comprise at least one modified nucleotide.
Paragraph 10. The method, kit, or signal probe of any proceeding Paragraphs, wherein the first nucleic acid sequence of the primer and/or the second nucleic acid sequence of the signal probe has/have 40 or fewer nucleotides.

### Measuring Location of Capture Probes

Variability in manufacturing can lead to false positives or false negatives. Additionally, a sample-to-answer system with variable detection limits may be able to detect a pathogen in one instance but not another. Thus, the ability to monitor and improve quality control during manufacturing is both a requirement and a best practice.

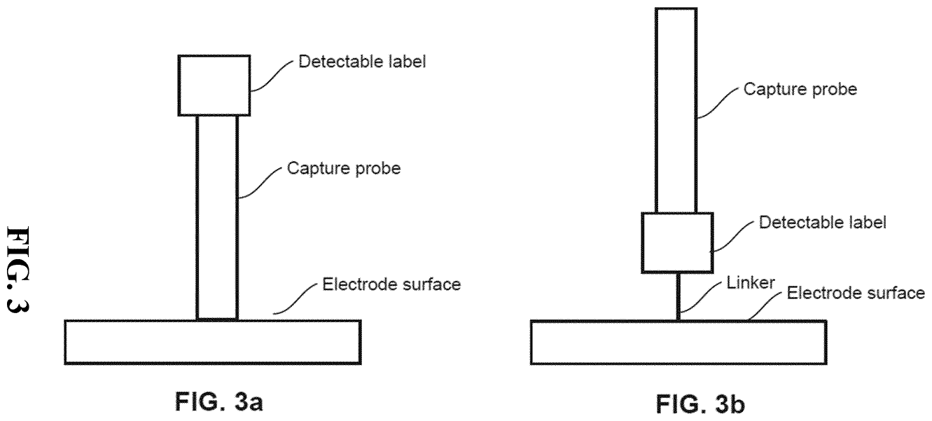

The detectable label on the capture probe can be an ETM or an optical signaling moiety. In some embodiments, a first plurality of detectable labels has an ETM, and a second plurality of capture probes have an optical label. The capture probe bound to a detectable label can be specific for the analyte of interest. The capture probe bound to a detectable label can serve as a control and may contain its own signal that is distinguishable from that which is bound to the signal probe. Stated another way, the labeled nucleic acid capture probes can be of the same or different redox potentials as the label on the signal probe. Stated another way, the labeled nucleic acid capture probes can be of the same or different modality as the label on the signal probe. In some instances, the signal probe can bind to the labeled capture probe and in some instances it cannot bind. In some embodiments the label on the capture probe and the first label on the signal probe are identical, but they need not be. In some embodiments, they are both energy transfer moieties. In some embodiments, they are both optical signaling moieties. In some embodiments they are ferrocene-based, ferrocene-derivative compounds, methylene blue or osmium. As shown in n FIG. 3a the detectable label, which can be an optical label, is at the free end of the capture probe. As shown in FIG. 3b the detectable label, which can be an optical label, is closer to the electrode surface and is connected to the electrode surface via a linker.

For example, FIG. 13 shows background fluorescence of the detection electrode/gold pad. Hardly any fluorescence is detected. FIG. 14, in contrast, shows nucleic acid stain on the electrode. Fluorescence is detected. And finally, FIG. 17 shows that the distribution of the capture probes can be visualized by hybridizing it to a signal probe comprising a fluorescent marker.
Paragraph 1. An electrode sensor device configured to evaluate capture probes deposited onto an electrode surface in order to determine if the electrode is within a first defined parameter and/or capture probe spotting device is within a second defined parameter, comprising:
   a capture probe spotting device;
   a capture probe comprising an optical signaling moiety;
   an electrode spotted with at least one capture probe wherein the electrode is configured to receive a voltage;
   an optical detector configured to detect the optical signaling moiety when excited by a voltage;
   a processor for analyzing the optical signaling moiety when excited by a voltage.
Paragraph 2. A method of demeaning if a detection electrode satisfies a defined parameter, the method comprising:
   spotting capture probes onto the detection electrode wherein the capture probe comprises an optical signaling moiety;
   applying a voltage to the capture probe;
   detecting a signal produced by the optical signaling moiety wherein if the signal produced by the optical signaling moiety is within a predetermined threshold, the detection electrode satisfies the defined parameter and can be incorporated into a detection cartridge.
Paragraph 3. A method of demeaning if a detection electrode spotting instrument satisfies a defined criterion, the method comprising:
   spotting capture probes onto the detection electrode wherein the capture probe comprises an optical signaling moiety;
   applying a voltage to the capture probe;
   detecting a signal produced by the optical signaling moiety wherein if the signal produced by the optical signaling moiety is within a predetermined range, the detection electrode spotting instrument satisfies the defined criterion and can be used to manufacture detection electrodes.
Paragraph 4. The method of Paragraphs 2 or 3 wherein the predetermined threshold and/or predetermined range comprises the location, density, concentration, or pattern of the capture probe.
Paragraph 5. The method of any of Paragraphs 2-4, further comprising determining a concentration of the capture probe spotted on the electrode surface
Paragraph 6. The method of Paragraph 5, further comprising, adjusting the detected signal produced by the optical signaling moiety based on the concentration of the capture probe spotted on the electrode surface.
Paragraph 7. The method of any of Paragraphs 2-6, further comprising repotting the electrode surface with capture probe if the location, density, concentration, or pattern of the capture probe is below the predetermined threshold and/or predetermined range.
Paragraph 8. The electrode sensor of Paragraph 1 further comprising a surface regenerator to clean the electrode surface if the location, density, concentration, or pattern of the capture probes are below a predetermined threshold and/or predetermined range.

### Location of Probes to Filter Out Probes in Detection Analysis

Thus, methods are developed that can achieve detection of spatial information (e.g., capture probe location, and/or distribution).

In some embodiments, certain probes signals can be filtered out, for example, probes on the periphery, probes that are located too close together, probes, which are not spotted uniformly etc. The filtering process examines the location of probes and disqualifies certain probes from the detection analysis.
Paragraph 1. A method of determining whether a target analyte is present in a sample the method comprising obtaining and storing location information for each capture probe on the electrode; determining whether a first probe should be filtered out of the detection analysis based on the stored location; selecting a subset of capture probes corresponding to probes having a desirable location; and determining whether the target analyte is present based on the selected subset of capture probes.
Paragraph 2. The method of Paragraph 1 wherein the step of determining comprises determining the probes' location on the electrode and/or in relation to other probes on the electrode.
Paragraph 3. The method of any prior paragraph, wherein the step of selecting a subset includes selecting all probes in a defined area.
Paragraph 4. The method of any prior paragraph further including the step of filtering the selected subset to remove outlying probe values prior to the determining step.
Paragraph 5. The method of any prior paragraph, wherein the step of filtering includes removing any probe having a signal two standard deviations from the normal distribution.
Paragraph 6. The method of any prior paragraph wherein the step of filtering includes determining an average signal of the probes and removing any probe having a signal more than a predetermined amount away from the determined average signal.
Paragraph 7. A system for determining whether a target analyte is present in a sample, the system comprising a location fetcher for obtaining and storing location information for a plurality of capture probes; a location processor for determining the location of each of the plurality of capture probes on an electrode; and a mapping engine for selecting a subset of the plurality of capture probes corresponding to probes having a desirable location on the electrode.
Paragraph 8. The system further comprising a filter for removing capture probe signals having a signal rating outside a predetermined rating.

### A Method for Calibrating a Sensor Assembly

A sensor assembly can measure various properties of deposited capture probes on the electrode surface, including position of capture probes, density of capture probes, patterns of capture probes etc. Sensed information is used to assure whether the electrode is manufactured properly, and whether the capture probe deposition values are within target ranges (parameters) so that the detection signal can be accurately relied upon to provide a detection result. The sensor assembly can also measure various metrics used to assess the status or “health” of the electrode. Various patterns of position, density or patterns of capture probes may indicate problems associated with the electrode as manufactured or the capture probe droplet generator (i.e., the device that deposits the capture probe onto the electrode).

In some embodiments, the electrochemical signal and the optical signal are transmitted simultaneously. In some embodiments, the electrochemical signal is received by the electrochemical sensor at the same time the optical signal is received by the optical sensor. In some embodiments, the correlation calculator receives the electrochemical signal from the electrochemical sensor at the same time as the optical signal is received from the optical sensor.

In some embodiments, the electrochemical signal and the optical signal are transmitted consecutively. In some embodiments, the electrochemical signal is received by the electrochemical sensor before or after the optical signal is received by the optical sensor. In some embodiments, the correlation calculator receives the electrochemical signal from the electrochemical sensor before or after the optical signal is received from the optical sensor.

### Kits

Disclosed are kits for the detection of nucleic acid molecules in a sample. Such kits may also be designed to detect nucleic acid molecules of interest during or after nucleic acid amplification reactions. Such kits may be diagnostic kits wherein the presence of the nucleic acid is correlated with the presence or absence of a disease or disorder. Disclosed are kits for carrying out amplification reactions described herein and to kits for making the compositions described herein. In specific embodiments, the kits comprise one or more dual-labeled signal probes as defined herein. The kit can further comprise additional components for carrying out the detection assays or other methods. Such kits may comprise one or more additional components selected from the group comprising, consisting of or consisting essentially of one or more polymerases (e.g., DNA polymerases and reverse transcriptases), one or more nucleotides, one or more buffering salts (including nucleic acid amplification buffers), one or more control nucleic acid target molecules (to act as positive controls to test assays), instructions for carry out the methods and the like.

### Kit For Detecting a SNP

In another embodiment, a kit for detecting a SNP in a target nucleic acid in a sample is provided, including at least one primer including a first nucleic acid sequence specific to produce an amplification product of the target nucleic acid; and a SNP specific dual-labeled signal probe comprising a second nucleic acid sequence complementary to a SNP containing region of the amplification product, the SNP specific signal probe comprising a first detectable label and a second detectable label (wherein the first detectable label and second detectable label are of different modalities). The kit may also include a polymerase enzyme having 5′ to 3′ exonuclease activity and a capture probe comprising a nucleic acid sequence complementary to a SNP containing region of the amplification product.

All references cited herein are incorporated by reference herein in their entirety.

1. A method of detecting the presence or absence of a target in a sample, wherein the method comprises the following steps:
(a) hybridizing a target analyte to a first signal probe to form a first signal probe-target hybridization complex, wherein the first signal probe has: (i) a first signaling moiety, and (ii) a second signaling moiety, and wherein the first signaling moiety and the second signaling moiety are detectable using different detection modalities;
(b) hybridizing the formed first signal probe-target hybridization complex to a first capture probe to form a first capture probe hybridization complex;
(c) exciting the first signaling moiety and the second signaling moiety by applying a voltage to the sample; and
(d) detecting the presence or absence of the target in the sample using electrochemical detection and optical detection.

2. The method of claim 1, wherein the first signaling moiety comprises an electron transfer moiety, and the second signaling moiety comprises an optical signaling moiety.

3. The method of claim 1, wherein the first signaling moiety of the signal probe comprises an electron transfer moiety label on the 5′ end, the 3′ end, or both the 5′ and 3′ ends.

4. The method of claim 1, wherein the second signaling moiety of the signal probe comprises an optical label on the 5′ end, the 3′ end, or both the 5′ and 3′ ends.

5. The method of claim 4, wherein the signal probe comprises an optical label, wherein the optical label is internal to the signal probe.

6. The method of claim 1, wherein the signal probe comprises a plurality of electron transfer moiety labels located at least at two different locations.

7. The method of claim 1, wherein the signal probe comprises: (i) a first portion, wherein the first portion is capable of hybridizing to the target analyte, (ii) a second portion, wherein the second portion is not capable of binding to the capture probe, and (iii) a third portion, wherein the third portion comprises the first signaling moiety and the second signaling moiety.

8. The method of claim 1, wherein the signal probe comprises: (i) a first portion, wherein the first portion is capable of hybridizing to the target analyte, (ii) a second portion, wherein the second portion comprises a linker, and (iii) a third portion, wherein the third portion comprises the first signaling moiety and the second signaling moiety, and wherein the linker couples the first portion and the third portion.

9. The method of claim 1, wherein the first signaling moiety is detected via electrochemical detection.

10. The method of claim 1, wherein the second signaling moiety is detected via optical detection.

11. The method of claim 1, wherein the first signaling moiety is ferrocene, a ferrocene derivative, or osmium.

12. The method of claim 1, wherein the first signaling moiety is methylene blue.

13. The method of claim 1, wherein the optical signal moiety is a fluorophore.

14. The method of claim 13, wherein the fluorophore is fluorescein.

15. A method of detecting the presence or absence of a target in a sample, wherein the method comprises the following steps:
(a) hybridizing a target analyte to a first signal probe to form a signaling complex, wherein the first signal probe has (i) a first signaling moiety, and (ii) a second signaling moiety, and wherein the first signaling moiety and the second signaling moiety are of a different modality;
(b) hybridizing the signaling complex with a first capture probe to form a capture probe hybridization complex;
(c) exciting the first signaling moiety and the second signaling moiety by application of a light or laser beam; and
(d) detecting the presence or absence of the target in the sample using electrochemical detection and optical detection.

16. The method of claim 15, wherein the first signaling moiety is an electrochemically detectable signaling moiety, and the second signaling moiety is an optical signaling moiety.

17. The method of claim 15, wherein the first signal probe cannot bind to the first capture probe.

18. The method of claim 15, wherein the signaling complex cannot bind to the first capture probe.

19. The method of claim 16, wherein a first portion of the signaling complex binds to the first capture probe, and a second portion of the signaling complex binds a second capture probe.

20. The method of claim 15, wherein the first signaling moiety is ferrocene, a ferrocene derivative, or osmium.

21. The method of claim 15, wherein the first signaling moiety is methylene blue.

22. The method of claim 15, wherein the optical signal moiety is a fluorophore.

23. The method of claim 22, wherein the fluorophore is fluorescein.

24. A method of detecting the presence or absence of a target in a sample, wherein the method comprises the following steps:
(a) hybridizing a target analyte to a first signal probe to form a signal probe-target hybridization complex;
(b) hybridizing the signal probe-target hybridization complex with a first capture probe to form a capture probe hybridization complex;
(c) exciting the first signal probe by applying a light or laser beam, and a voltage; and
(d) detecting the presence or absence of the target in the sample using electrochemical detection and optical detection.

25. The method of claim 24, wherein the first signal probe comprises: (i) a first signaling moiety, wherein the first signaling moiety is an electron transfer moiety, and (ii) a second signaling moiety, whichwherein the second signaling moiety is a Green Fluorescent Protein (GFP).

26. The method of claim 24, wherein the first signaling moiety is detected via electrochemical detection.

27. The method of claim 24, wherein the second signaling moiety is detected via optical detection.

28. The method of claim 24, wherein the first signaling moiety is not excited via application of a voltage.

29. A signal probe comprising: (a) a first signaling moiety; and (b) a second signaling moiety, wherein the first signaling moiety and the second signaling moiety are detectable through different detection modalities.

30. The signal probe of claim 29, wherein the first signaling moiety is ferrocene, a ferrocene derivative, or osmium.

31. The signal probe of claim 29, wherein the first signaling moiety is methylene blue.

32. The signal probe of claim 29, wherein the second signaling moiety is a fluorophore.

33. The signal probe of claim 32, wherein the fluorophore is fluorescein.
